(19) United States
(12) Patent Application Publication
(10) Pub. No.: US 2022/0233602 A1
(43) Pub. Date: Jul. 28, 2022

(54) GENERATION OF NEURAL STEM CELL LINES DERIVED FROM HUMAN PLURIPOTENT STEM CELLS

(71) Applicant: Novo Nordisk A/S, Bagsvaerd (DK)

(72) Inventors: Juan Carlos Villaescusa Ramirez, Koebenhavn S (DK); Ulrik Doehn, Oelstykke (DK); Charlotte Vinther Bertelsen, Koebenhavn S (DK)

(21) Appl. No.: 17/617,468

(22) PCT Filed: Jul. 1, 2020
(86) PCT No.: PCT/EP2020/068590

§ 371 Date: Dec. 8, 2021

(30) Foreign Application Priority Data
Jul. 5, 2019 (EP) 19184630.2
Dec. 23, 2019 (EP) 19219351.4

(51) Int. Cl. A61K 35/30; C12N 5/0797

(52) U.S. Cl. CPC A61K 35/30; C12N 5/0623; C12N 2501/15; C12N 2533/52; C12N 2506/02; C12N 2513/00; C12N 2501/727

(57) ABSTRACT

The present invention relates generally to the field of stem cells, more specifically to highly pure neural stem cell population, a method for obtaining such highly pure stem cell-derived line of neural stem cells, such as a neural stem cell line derived from pluripotent stem cells, such as human embryonic stem cells. Furthermore, the present invention relates to use of such highly pure neural stem cell lines for use as a medicament for use in the treatment of neurodegenerative diseases.

### TECHNICAL FIELD

The present invention relates generally to the field of stem cells, more specifically to a highly pure neural stem cell population, a method for obtaining such highly pure stem cell-derived line of neural stem cells, such as a neural stem cell line derived from pluripotent stem cells, such as human embryonic stem cells. Furthermore, the present invention relates to use of such highly pure neural stem cell lines, for use as a medicament for use in the treatment of neurodegenerative diseases and use of exosomes obtained from such neural stem cells for the treatment of stroke.

### BACKGROUND

Neural stem cells (NSCs) are self-renewing, multipotent cells with the capacity of generating neurons and glia cells. NSCs are temporarily present during normal embryonic development of the central nervous system, and similar cells can also be derived from e.g. human pluripotent stem cells (hPSCs). To date, extensive efforts have been made for the development of efficient protocols for the successful differentiation of hPSCs into populations of highly pure and expandable human NSCs. Well-established methods for the differentiation of hPSCs into human NSCs are via the formation of cellular aggregates, so called embryoid bodies. However, many current protocols lead to heterogeneous cell populations, comprising, for example, side cellular populations of non-neural identity. Hence, manual selection of NSCs (for example by manual picking of neural rosettes) is required, which is a laborious and time-consuming process, thereby limiting scalability.

Over the last years, it has been explored the use of NSC-derived extracellular vesicles (EVs) and exosomes to treat cerebral damage and neurodegeneration, including cerebral infarctions. Extracellular vesicles (EVs) are lipid bilayer-delimited particles that are naturally released from a cell. EVs range in diameter from near the size of the smallest physically possible unilamellar liposome (around 20-30 nanometers) to as large as 10 microns or more, although the vast majority of EVs are smaller than 200 nm. Exosomes are nanoscale EVs (from 30-150 nm in diameter) released by most cell types (Colombo et al., 2014). Exosomes contain a variety of molecules (cargo) that can comprise proteins, nucleic acids and lipids derived from their host cells, facilitating intercellular communication and regulating recipient cell function (Robbins et al., 2016). Recent work utilizing exosomes for ischemic stroke therapy have attributed improved functional outcome to their cargoes, which includes microRNAs, DNAs, lipids, proteins and RNAs (Chen and Chopp, 2018). These observations suggest that application of NSC-derived exosomes may be a promising new avenue for the treatment of brain damage, including stroke and traumatic brain injury (TBI).

A few documents (Ruttachuk et al. 2013, Watanabe et al. 2007) allegedly disclose methods for obtaining NSCs from hPSCs in 2D cultures using ROCKi and/or dual SMAD inhibition (Qiuhong et al. 2019, US2017260501, Meixiang et al. 2018). In some of these methods, dissection prior to expansion is required to increase purity, i.e., manual selection and isolation of the cells. This is time and resource consuming and reduces the capability for scalability of said methods.

It is an object of the present invention to overcome the aforementioned challenges, in particular to provide a method for obtaining NSC lines, which enables scalability and provides a GMP population of NSCs with very high purity.

### SUMMARY

The aforementioned objects are achieved by the aspects of the present invention. In addition, the present invention may also solve further problems, which will be apparent from the disclosure of the exemplary embodiments. In a first aspect of the present invention is provided a method for obtaining in vitro NSC lines from pluripotent stem cells (PSCs), comprising the steps of dissociating the PSCs into single cells, culturing the PSCs in a suspension culture, allowing the PSCs in suspension to spontaneously form tridimensional cellular aggregates and differentiating them into neuroectodermal spheres (NECS) consisting of neuroectodermal cells including NSCs, optionally dissociating the NSCs, plating the NSCs on a substrate, allowing the NSCs to form neural rosettes, and maintaining and expanding the NSCs to establish the NSC lines.

The protocol used for neural induction of PSCs and subsequent generation and establishment of a NSC line can roughly be divided in three main stages: a neuroectoderm induction and NECS formation; NECS plating and rosette formation, and finally NSC line establishment by replating the cells in expansion media. By following these main stages, the present inventors have developed a method for enabling a fast and reliable NECS-based differentiation protocol, which yields a largely pure and expandable human NSC population with neural rosette morphology. The method facilitates differentiation and establishment of NSC lines. After ten days the present inventors have observed clear and visible neural rosettes in 2D, which were ready to dissociate. The NSC line is established when the neural rosettes are dissociated and replated for an additional 3-4 passages. One major advantage is the avoidance of the tedious and time-consuming step of manual isolation. The present inventors believe that controlling the formation of uniform NECS without forced aggregation enables the method to provide such high purity. Accordingly, the present inventors found that the step of allowing the PSCs to spontaneously form NECS is of particular importance. The spontaneous formation in part facilitates a population of NECS with a size that, once plated, result in a highly pure, well-defined and homogeneous population of neural rosettes. In a preferred embodiment, the method further comprises a step of subjecting the suspension culture to agitation. The present inventors have surprisingly found that after an initial non-forced formation of the NECS, subjecting the culture to agitation furthers the establishment of a population of the NECS with a diameter size of less than 500 μm. It has further been surprisingly found that by controlling the size of the NECS one can improve the purity and formation of the neural rosettes. In a preferred embodiment, the diameter of the NECS is less than 500 μm prior to the step of plating the NSCs.

In one aspect, the present invention relates to a method for obtaining neuroectodermal cells from pluripotent stem cells (PSCs) comprising the steps of:
contacting said PSCs with ROCKi and a single SMAD inhibitor in suspension culture,
allowing said PSCs in suspension to spontaneously form tridimensional cell aggregates,
allowing said tridimensional cell aggregates to differentiate into neuroectodermal spheres with a diameter of less than 500 μm in dynamic cell culture suspension,
allowing said neuroectodermal spheres to form neural rosettes, wherein said neural rosettes comprise neuroectodermal cells.

In one aspect, the present invention relates to a method for obtaining neuroectodermal cells from pluripotent stem cells (PSCs) comprising a step of culturing said PSCs in a medium comprising a single SMAD inhibitor.

In another aspect, the present invention relates to a method for obtaining neuroectodermal cells from pluripotent stem cells comprising a step of culturing said PSCs in a medium comprising a single SMAD inhibitor, wherein said single SMAD inhibitor is RepSox or GW788388.

In one aspect, the present invention relates to a method for obtaining neuroectodermal cells from pluripotent stem cells comprising a step of culturing said pluripotent stem cells in a medium comprising a single SMAD inhibitor, wherein said single SMAD inhibitor is RepSox in the concentration of about 20 μM to about 60 μM.

In one aspect, the present invention relates to a method for obtaining neuroectodermal cells from pluripotent stem cells comprising a step of culturing said pluripotent stem cells in a medium comprising a single SMAD inhibitor, wherein said single SMAD inhibitor is GW788388 in the concentration of about 0.1 ng/ml to about 150 ng/ml.

Another aspect of the present invention relates to method for obtaining neuroectodermal cells from PSCs, comprising the steps of contacting the PSCs with an inhibitor of the Transforming Growth Factor (TGF)/activin/nodal signaling pathway, and allowing the PSCs to differentiate into neuroectodermal cells, wherein the inhibitor of the Transforming Growth Factor (TGF)/activin/nodal signaling pathway is RepSox, preferably in a concentration ranging from about 0.1 μM to about 100 μM. The present inventors have surprisingly found that this small molecule at certain concentrations facilitates the differentiation of PSCs into the neuroectodermal lineage with a very high efficiency, even without the need for simultaneous inhibition of the bone morphogenetic protein (BM P) signaling pathway, i.e. without dual SMAD inhibition, such as contacting the PSCs with Noggin. This simplifies differentiation protocol into the neuroectoderm lineage and facilitates translation of protocols into GMP compliance.

In one aspect, the present invention relates to a highly pure neural stem cell population, wherein said neural stem cells are at least 80% double positive for OTX2/PAX6 or PAX6/SOX2.

In one aspect, the present invention relates to a highly pure neural stem cell population, wherein said neural stem cells are at least 80% triple positive for OTX2/PAX6/SOX2.

In another aspect of the present invention is provided the use of the NSC lines of the present invention for producing exosomes. In a further aspect is provided a method for producing exosomes from the NSC lines obtained according to the methods of the present invention, comprising the steps of allowing the NSCs to produce exosomes and isolating the exosomes.

Another aspect of the present invention relates to exosomes for use as a medicament, specifically for the use in the treatment of a neurodegenerative disorder and/or brain injury, such as but not limited to stroke, traumatic brain injury (TBI) and Alzheimer's disease. Accordingly, in one embodiment the neurodegenerative disorder is stroke. In another embodiment, the neurodegenerative disorder is traumatic brain injury (TBI). In yet another embodiment, the neurodegenerative disorder is Alzheimer's disease.

In another aspect of the present invention is provided a method of maintaining and expanding a NSC line comprising the steps of culturing NSCs on a substrate, allowing the NSCs to reform neural rosettes, dissociating the NSCs into single suspension, contacting the NSCs with a ROCK inhibitor, and replating the NSCs on a second substrate. The present inventors surprisingly found that contacting the NSCs with a ROCK inhibitor at each passage significantly maintain the complexity of the rosette structure, morphology and neural precursor composition.

In one aspect, the present invention relates to a method for obtaining an in vitro neural stem cells, comprising the steps of:
dissociating the PSCs into single cells,
contacting said PSCs with ROCKi and a single SMAD inhibitor in suspension culture,
allowing the PSCs in suspension to spontaneously form tridimensional cell aggregates
allowing said tridimensional cell aggregates to differentiate into neuroectodermal spheres with a diameter of less than 500 μm in dynamic cell culture suspension,
plating the NECS-containing neuroectodermal cells on a substrate or optionally dissociating the NECS-containing NSCs,
allowing the NSCs to form neural rosettes and maintaining and expanding the NSCs to establish the NSC lines, without the need of manual picking and isolation.

In another aspect, the present invention relates to a method for obtaining in vitro neural stem cell (NSC) lines from PSCs, comprising the steps of:
dissociating said PSCs into single cells or aggregates comprising less than about 50 cells,
culturing said obtained cells in a dynamic suspension culture,
allowing said obtained cells in suspension to spontaneously form NECS, and further generate NSCs.
allowing the NSCs to be maintained and expanded to establish the NSC lines.

The advantage of the method according to this last aspect is the fast provision of NSCs readily available for further differentiation into specific cells of the neuroectodermal lineage either by culturing in 2D or in suspension.

It applies throughout the application that in a preferred embodiment of the aforementioned aspects the PSCs are hPSCs. In follows that also the product in a preferred embodiment is human-derived. Accordingly, in a preferred embodiment the NSCs and NSC lines are human NSCs and NSC lines, respectively.

### BRIEF DESCRIPTION OF DRAWINGS

FIG. 1 shows a schematic representation of the experimental protocol. “Tested compound” refers to any of the small molecules tested: SB431542, LDN, GW788388, RepSox, SB525334, LY2157299, TEW-7197 or LY2109761. “Y” stands for 10 μM ROCKi (Y-27632).

FIG. 2 shows bright fields microscopy photos taken at day 3 clearly shows that shaking improves uniformity of NECS. Scale bar: 200 μm.

FIG. 3 shows the morphology of NECS from culture with GW788388 and RepSox. The small molecule RepSox induce the formation of NECS with bright cavities, whereas GW788388 results in a darker and denser core.

FIG. 4 shows bright field microscopy photos of NECS in suspension and adherent NECS at different timepoints during the process. At day 3, several NECS are formed in a suspension culture. After seeding in 2D many rosettes are visible at day 8 and several uniform rosettes at day 10. Scale bar: 200 μm (A, E); 100 μm (F); 50 μm (B, C, D).

FIG. 5 shows an example of neural rosette formation using RepSox (25 uM), showing apical localization of ZO1 protein, located in the center (lumen) of each neural rosette. All cells were positive for the neural progenitor marker Nestin (NES).

FIG. 6 shows a comparison of the two chemical compounds, GW788388 and RepSox. Cells are stained with the NSC marker NES along the tight junction marker ZO1, thus, allowing visualization of neural rosettes and other structures. While multiple neural rosettes are observed when hESCs are treated with RepSox, only few rosettes are observed with GW788388. Additionally, cells non-forming neural rosettes are observed with GW788388. Scale bar: 100 μm.

FIGS. 7 and 8 show cells that are stained with the NSC marker NES along the tight junction marker ZO1, and counterstained with DAPI at RepSox 0.25 μM, 2.5 μM, 25 μM, and 50 μM, respectively. The organization of cells are clearly affected by the amount of RepSox, where higher doses improves neural rosette formation. White arrows indicate NES negative cells.

FIGS. 9 and 10 show variation in neural rosette size between biological replicates with RepSox 50 μM. Scale bar: 100 μm.

FIG. 11 show the NECS size distribution of each replication. The graph shows the number of NECS (Particle Count) into an interval of relative NECS diameter, with 50 units increments (in μm).

FIG. 12 show the effect of a NECS bigger than 400 μm. Not all the cell (DAPI) are positive for the marker NES.

FIG. 13 shows expandability and the population doubling time (in days) of NSC line generated by the presented method. Additionally, a linear regression model has been applied to the log 2-transformed data to visualize how the NSCs expand and doubles over time through several cell passages.

FIGS. 14 and 15 show different passages of the established NSC line showing retained neural rosette formation (NES/ZO-1) after 5 passages. Additionally, all cells are positive for SOX2, and almost all cells are positive for PAX6 and OTX2. Scale bar: 100 μm.

FIG. 16 shows passage number 12 of the established NSC line showing retained neural rosette formation (NES/ZO-1). Additionally, all cells are positive for SOX2, and almost all cells are positive for PAX6 and OTX2. Scale bar: 100 and 10 μm.

FIG. 17 shows passage 12 of the established NSC line show retained neural rosette formation and positive for the anterior markers FOXG1 and OTX2. Scale bar: 100 μm.

FIG. 18 shows relative gene expression for NSC relative to hESC. Left axis shows the relative fold change to hESC calculated by the 2-ΔΔCt method. Right axis is −ΔΔCt, and presented values are mean of ΔCts normalized to hESC (ΔΔCt)±SD. 25 μM (n=5), 50 μM (n=4). Genes are grouped in markers typical for pluripotency, NSC and neuron-restricted progenitor, anterior/forebrain, midbrain and posterior/hindbrain. Statistical test is performed on log 10(dCt) values with a significance level of α=5% (p<0.01 (**) and p<0.001 (***)). N.d.=not detected or Ct>35.

FIG. 19 shows the quantification of double positive cells at passage 3, for the two neural stem cell markers, PAX6 and OTX2, determined by flow cytometry (FACS). The percentage of PAX6/OTX2 positive cells is 93.4%.

FIG. 20 shows the quantification of double positive cells at passage 8, for the neural stem cell markers, PAX6, OTX2 and SOX2, and the forebrain maker FOXG1 determined by flow cytometry (FACS). The percentages of PAX6/OTX2, PAX6/FOXG1 and PAX6/SOX2 are above 80%.

FIG. 21 shows exosomes are present in supernatant collected from both hESC and NSC. Size, protein content and number of particles differs between exosomes produced by hESC and by NSC. The structure of exosomes produced by NSC is imaged by electron microscopy.

### DESCRIPTION

Unless otherwise stated, all technical and scientific terms used herein have the same meanings as commonly understood by one of ordinary skill in the art to which this invention belongs. The practice of the present invention employs, unless otherwise indicated, conventional methods of chemistry, biochemistry, biophysics, molecular biology, cell biology, genetics, immunology and pharmacology, known to those skilled in the art.

It is noted that all headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Throughout this application the terms “method” and “protocol” when referring to processes for differentiating cells may be used interchangeably. The methods of the present invention are typically defined by a series of steps. As used herein, the term “step” in relation to the methods is to be understood as a stage where something is undertaking and/or an action is performed. It will be understood by one of ordinary skill in the art when the steps to be performed and/or the steps undertaking are concurrent and/or successive and/or continuous.

As used herein, “a” or “an” or “the” can mean one or more than one. Unless otherwise indicated in the specification, terms presented in singular form also include the plural situation. As used herein, “and/or” refers to and encompasses any and all possible combinations of one or more of the associated listed items, as well as the lack of combinations when interpreted in the alternative (“or”). Moreover, the present invention also contemplates that in some embodiments of the invention, any feature or combination of features set forth herein can be excluded or omitted.

The term “about,” as used herein when referring to a measurable value such as an amount of cells, a compound or an agent of this invention, dose, temperature, and the like, is meant to encompass variations of 5%, 1%, 0.5%, or even 0.1% of the specified amount. As used herein, the term “day” in reference to the protocols refers to a specific time for carrying out certain steps.

In general and unless otherwise stated “day 0” refers to the initiation of the protocol, this be by for example but not limited to plating the stem cells or transferring the stem cells to an incubator or contacting the stem cells in their current cell culture medium with a compound prior to transfer of the stem cells. Typically, the initiation of the protocol will be by transferring undifferentiated stem cells to a different cell culture medium and/or container such as but not limited to by plating or incubating, and/or with the first contacting of the undifferentiated stem cells with a compound that affects the undifferentiated stem cells in such a way that a differentiation process is initiated.

When referring to “day X”, such as day 1, day 2 etc., it is relative to the initiation of the protocol at day 0. One of ordinary skill in the art will recognize that unless otherwise specified the exact time of the day for carrying out the step may vary. Accordingly, “day X” is meant to encompass a time span such as of +/−10 hours, +/−8 hours, +/−6 hours, +/−4 hours, +/−2 hours, or +/−1 hours.

As used herein, the phrase “from at about day X to at about day Y” refers to a day at which an event starts from. The phrase provides an interval of days on which the event may start from. For example, if “cells are contacted with a differentiating factor from at about day 3 to at about day 5” then this is to be construed as encompassing all the options: “the cells are contacted with a differentiating factor from about day 3”, “the cells are contacted with a differentiating factor from about day 4”, and “the cells are contacted with a differentiating factor from about day 5”. Accordingly, this phrase should not be construed as the event only occurring in the interval from day 3 to day 5. This applies mutatis mutandis to the phrase “to at about day X to at about day Y”.

Hereinafter, the methods according to the present invention are described in more detail by non-limiting embodiments and examples. A method is provided for obtaining neural stem cell lines from PSCs. Accordingly, the method takes offset in the use of stem cells.

By “stem cell” is to be understood as an undifferentiated cell having differentiation potency and proliferative capacity (particularly self-renewal competence) but maintaining differentiation potency. The stem cell includes subpopulations such as pluripotent stem cell (PSC), multipotent stem cell, unipotent stem cell and the like according to the differentiation potency.

As used herein, the term “pluripotent stem cell” (PSC) refers to a stem cell capable of being cultured in vitro and having a potency to differentiate into any cell lineage belonging to three germ layers (ectoderm, mesoderm, endoderm). A PSC can be induced from fertilized egg, clone embryo, germ stem cell, stem cell in a tissue, somatic cell and the like. Examples of the PSC include embryonic stem cell (ESC), induced pluripotent stem cell (iPSC), embryonic germ cell (EG cell) and the like. Muse cell (Multi-lineage differentiating stress enduring cell) obtained from mesenchymal stem cell (MSC), and germline stem cell (GS cell) produced from reproductive cell (e.g., testis) are also encompassed in the PSC term. The pluripotent stem cells used in the present invention can thus be embryonic stem cells prepared from blastocysts, as described in e.g. WO 03/055992 and WO 2007/042225, or be commercially available cells or cell lines. ES cell lines can also be derived from single blastomeres without the destruction of ex utero embryos and without affecting the clinical outcome (Chung et al. (2006) and Klimanskaya et al. (2006)).

As used herein, the term “induced pluripotent stem cell” (also known as iPS cells or iPSCs) means a type of PSC that can be generated directly from adult cells by a process commonly known as reprogramming. By the introduction of products of specific sets of pluripotency-associated genes adult cells can be converted into PSCs. Embryonic stem cells may also be derived from parthenotes as described in e.g. WO 2003/046141. Additionally, embryonic stem cells can be produced from a single blastomere or by culturing an inner cell mass obtained without the destruction of the embryo. Embryonic stem cells are available from given organizations and are also commercially available. Preferably, the methods and products of the present invention are based on hPSCs, i.e. stem cells derived from either iPSCs or embryonic stem cells, including parthenotes.

The present invention provides a method for obtaining in vitro NSC lines from PSCs, comprising the steps of
dissociating the PSCs into a single cells,
culturing obtained cells in a suspension culture,
allowing said obtained cells in dynamic suspension to spontaneously form NECS, differentiating said NECS into NSCs, plating the NSCs on a substrate or optionally dissociating the NSCs,
allowing the NSCs to form neural rosettes and maintaining and expanding the NSCs to establish the NSC lines.

As used herein, the term “neural” refers to the nervous system. As used herein, the term “neural cell” refers to cells mimicking a cell type, which is naturally part of the ectoderm germ layer, more specifically the neuroectoderm and the term is meant to encompass cells at any stage of development within this germ layer, such as early neural progenitor cells all the way through to mature, post-mitotic neurons.

As used herein, the term “neuroectodermal cell” refers to cells at any stage during the development along the neuroectodermal lineage.

As used herein, the term “neural stem cell” (NSC) refers to multipotent cells which are able to self-renew and proliferate without limit, to produce progeny cells which may terminally differentiate into neural and glial cells, such as neurons, astrocytes and oligodendrocytes. The non-stem cell progeny of NSCs are referred to as neural progenitor cells.

As used herein, the terms “neural precursor cell” and “neural progenitor cell” may be used interchangeably and mean a cell further derived from a neural stem cell, but without maintaining the extensive proliferative capacity.

As used herein, the term “neural stem cell line” means a population of NSCs that can be passaged for at least 10 passages maintaining the neural rosette structure and the markers ZO1, NES, SOX2, OTX2 and PAX6. By referring to a method for obtaining neural NSC lines is meant that one may establish one or more NSC lines. The present inventors have observed neural rosette formation in 2D already after ten days. The NSC line is established when the neural rosettes are dissociated and replated for an additional 3-4 passages. Hereafter the NSC line is considered established from which point the NSCs can be passaged for at least 10 passages. In one embodiment, the NSC line is not immortalized, i.e. in one embodiment the NSC line can be passaged for no more than 100 passages.

In one embodiment, the established NSC line of the present invention is passaged for 3-4 passages. In one embodiment, the established NSC line of the present invention is passaged for 10 passages. In one embodiment, the established NSC line of the present invention is passaged for 20 passages.

By the term “in vitro” is meant that the NSCs are provided and maintained outside of the human or animal body. In an embodiment, the NSCs are non-native.

By the term “non-native” is meant that the NSCs although derived from PSCs which may have human origin is an artificial construct that does not exist in nature. In general, it is often an object within the field of stem cells to provide cells that resemble the cells of the human body as much as possible. However, it may never become possible to mimic the development which PSCs undergo during the embryonic and fetal stage to such an extent that the mature cells are indistinguishable from native cells of the human body. Inherently, in an embodiment of the present invention, the NSCs are artificial.

As used herein, the term “artificial” may comprise material naturally occurring in nature but modified to a construct not occurring naturally. This includes human stem cells, which are differentiated into non-naturally occurring cells mimicking the cells of the human body. Preferably, the NSCs are stem cell-derived. More preferably, the NSCs are stem cell-derived from PSCs. In a further embodiment, the NSCs are stem cell-derived from human embryonic stem cells (hESCs) and/or human induced pluripotent stem cells (hiPSCs).

The PSCs are initially dissociated into single cells. As used herein, the term “dissociating into single cells” means bringing the PSCs into a single cell suspension. By “single cell suspension” is meant a cell suspension or suspension culture in which single cells and/or tridimensional small aggregates of cells typically of less than about five or six cells are allowed to function and multiply. When bringing cells in suspension the majority of cells are not adherent to a substrate or container surface. Any suitable means for bringing the PSCs into single cell suspension may be used. A person skilled in the art will readily recognize that several methods exist for bringing PSCs into single cell suspension, such as enzymatically or chelating. In an embodiment, the PSCs are dissociated into single cell suspension at day 0. Bringing the PSCs into suspension means subjecting the cells to a treatment that facilitates the dissociation of the cells from each other, from a substrate and/or an extracellular matrix. In an embodiment, the PSCs are dissociated into single cell suspension by contacting the PSCs with a cell dissociation agent, such as trypsin and/or TrypLE Select.

In a further step, the PSCs are cultured in a suspension culture. As used herein, the term “suspension culture” means that single cells or aggregates of cells are floating freely in a liquid medium. Suspension culturing may also be referred to as three-dimensional culturing and the two terms may be used interchangeably throughout the application. Accordingly, the PSCs are not attached to a substrate surface or otherwise fixed in a scaffold, such as an extracellular matrix. However, it is well recognized that some cells in a suspension culture may attach to the surface of the vessel. Without agitation of the suspension medium the cells will eventually settle on the surface of the vessel due to gravitational forces.

The term “culturing” is to be understood a process by which the stem cells are grown under controlled conditions, generally outside their natural environment as a continuous procedure, which may by employed throughout the method in order to maintain the viability of the cells at their various stages. After the cells of interest have been isolated from, for example but not limited to, living tissue or embryo, they are subsequently maintained under carefully controlled conditions. These conditions vary for each cell type, but generally consist of a suitable vessel with a medium that supplies the essential nutrients (amino acids, carbohydrates, vitamins, minerals), growth factors, hormones, and gases (CO₂, O₂), and regulates the physio-chemical environment (pH buffer, osmotic pressure, temperature).

Typically, the stem cells will be provided in a cell culture medium, which is suitable for viability in their current state of development. Providing the stem cells for culturing typically implies a transfer of the stem cells into a different environment such as by seeding onto a new substrate or suspending in an incubator. One of ordinary skill in the art will readily recognize that stem cells are fragile to such transfer and the procedure requires diligence and that maintaining the stem cells in the origin cell culture medium may facilitate a more sustainable transfer of the cells before replacing the cell culture medium with another cell culture medium more suitable for the differentiation process. In one embodiment, the cell culture medium at day 0 is a first cell culture medium and at least part of the cell culture medium is replaced with a second cell culture medium from day 1.

As used herein, the term “replacing” in reference to cell culture medium, first cell culture medium, and second cell culture medium means a procedure, wherein an amount of cell culture medium is taken out by suitable means, and, optionally, a substantially equal amount of cell culture medium is added so that the total volume of cell culture medium substantially remains the same. By “removing the first cell culture medium” is to be understood as after a first removal and addition of the second cell culture medium then any subsequent replacement will be a replacement of a mixture of the first and second cell culture medium, the mixture being in the ratio corresponding to the amounts removed and added. Accordingly, in a sequential removal, the first cell culture medium will be continuously diluted by the second cell culture medium and by repeating this procedure the cell culture medium eventually will be substantially free of the first cell culture medium.

In a further embodiment the cell culture media are chemically defined and xeno-free. As used herein, the term “chemically defined” in reference to a cell culture medium means a growth medium suitable for the in vitro cell culture of human or animal cells in which all of the chemical components are known. The chemically defined media require that all of the components must be identified and have their exact concentrations known.

As used herein, the terms “xeno-free” and “animal-free” may be used interchangeably and according to the present invention mean preferably completely devoid of any animal-derived components. In a preferred embodiment, the cell culture medium is also feeder-free.

The terms “feeder-free” and “feeder cell-free” may be used interchangeably and refer to the culturing system being devoid of human and animal cells which may be otherwise present for the purpose of nourishing the cultured stem cells, i.e. the feeder cells supply metabolites to the stem cells they support but are not the cells intended for growth or division. Even though the present inventors prefer a chemically defined, “xeno-free” and “feeder cell-free” cell culturing environment, regulatory bodies may approve medicinal products and treatments based on the methods according to the present invention without fully complying with such standard. The present inventors endeavor to adhere to the highest standards of GMP and GTP. However, the present invention should not be construed as limited to such standards. A person skilled in the art will readily acknowledge that the present invention may be carried out without adhering to such high standards.

In an embodiment a first cell culture medium may be any suitable cell culture medium which supports viability of the stem cells upon transfer to the substrate. Such cell culture media are commercially available and could for instance be Nutristem®, such as Nutristem® hPSC XF Medium for iPS and ES Stem Cells. Accordingly, in an embodiment the Nutristem®, such as Nutristem® hPSC XF Medium for iPS and ES Stem Cells.

In an embodiment a second cell culture medium is chemically defined and xeno-free. In a further embodiment, the second cell culture medium is also feeder-free. In an embodiment the second cell culture medium comprises GMEM, DMEM, or DMEM/F12. Similar media may work equally well and are readily available for purchase. In a further embodiment the DMEM/F12 is supplemented with N₂ and/or B27. In an embodiment, the concentration of N2 from about 0.01% (v/v) to about 5% (v/v), preferably from about 0.5% (v/v) to about 2.5% (v/v). In one embodiment, the concentration of B27 from about 0.05% (v/v) to about 1% (v/v), preferably about 0.1% (v/v).

In an embodiment, the PSCs are contacted with a ROCK inhibitor in the step of dissociating the PSCs into single cell suspension.

### ROCK Inhibitor

Rho-associated coiled-coil containing kinases (ROCK) is an effector of the RhoA small GTPase and belongs to the AGC family of serine/threonine kinases. ROCK kinases have many functions including cell contraction, migration, apoptosis, survival, and proliferation. IRho-associated, coiled-coil containing protein kinase ROCK inhibitors are a series of compounds that target and inhibit rho kinase. As used herein, “Y-27632” refers to trans-4-(1-Aminoethyl)-N-(4-Pyridyl) cyclohexanecarboxamide di hydrochloride with CAS no. 129830-38-2.

In one embodiment, the cell culture medium comprises a ROCK inhibitor. In one embodiment the ROCK inhibitor is Y-27632 or Tiger.

Accordingly, in a preferred embodiment, the PSCs are contacted with the ROCK inhibitor at the same time as the PSCs are dissociated into single cell suspension. In an embodiment, the concentration of the ROCK inhibitor is from about 0.5 μM to about 50 μM, preferably from about 5 μM to about 25 μM, even more preferably about 10 μM. In an embodiment, the concentration of the ROCK inhibitor is gradually reduced from about day 1.

It follows that in an embodiment, the PSCs are contacted with the ROCK inhibitor for about one day starting from the step of dissociating the PSCs into single cell suspension at day 0 at a concentration of from about 0.5 μM to about 50 μM, preferably from about 5 μM to about 25 μM, even more preferably about 10 μM. In one embodiment the ROCK inhibitor is Y-27632. The method comprises a step of allowing the PSCs in single cell suspension to spontaneously form NECS.

### Tridimensional Cell Aggregates

As used herein, the term “tridimensional cell aggregates” means a cluster of stem cells which is formed from a single cell, or an aggregate of a few cells attached to each other for a short time, i.e., 1-2 days. The tridimensional cell aggregate is formed through an initial cell attachment between few PSCs and/or few cell divisions of the cells and inherently grows as division continues. For this process, the initial PSC suspension culture is exposed to ROCK inhibitor. The step of allowing the PSCs to spontaneously form tridimensional cell aggregates and subsequently bigger cell aggregates that form NECS is a passive procedure, in the presence of ROCKi. As used herein, the term “spontaneously” when referring to the formation of tridimensional cell aggregates means that the formation of the tridimensional cell aggregates as such is not forced or facilitated by any means apart from bringing the PSCs into single cell suspension.

In one embodiment, the tridimensional cell aggregates are formed following exposure of PSCs to ROCKi for 24 hours. In one embodiment, the tridimensional cell aggregates are formed following exposure of PSCs to ROCKi for the first 24 hours. In one embodiment, the tridimensional cell aggregates are formed following exposure of PSCs to ROCKi for at least 24 hours. In one embodiment, the tridimensional cell aggregates are formed following exposure of PSCs to ROCKi for about 1-3 days. In one embodiment, the tridimensional cell aggregates are formed following exposure of PSCs to ROCKi for about 2-3 days. In one embodiment, the tridimensional cell aggregates are formed following exposure of PSCs to ROCKi for about 1 day. In one embodiment, the tridimensional cell aggregates are formed following exposure of PSCs to ROCKi for about 2 days. In one embodiment, the tridimensional cell aggregates are formed following exposure of PSCs to ROCKi for about 3 days.

### Neuroectoderm Spheres

As used herein, the term “neuroectoderm spheres” (NECS) means tridimensional cell aggregates that has grown in size due to cell division and at the same time differentiated into the neuroectodermal fate for several days, i.e., 5-6 days. According to the method of the present invention the cells of the NECS are directed towards the neuroectoderm lineage from the onset, when forming the initial small tridimensional cell aggregates. NECS will contain neuroectodermal cells, NSCs and NSCs forming neural rosettes depending on the phase of maturation. The NECS is formed through cell division of the cells and inherently grows as division continues. It follows that in an embodiment, the PSCs substantially form NECS via mitosis. As used herein, the term “mitosis” refers to cell division giving rise to genetically identical cells in which the number of chromosomes is maintained. In the method according to the present invention the NECS are formed by the PSCs dividing while differentiating. Accordingly, the NECS initially comprises PSCs and as these cells divide and undergo differentiation, they form NECS that comprises differentiated stem cells. The step of allowing the PSCs to spontaneously form small cell aggregates and subsequently bigger NECS is a passive procedure. As used herein, the term “spontaneously” when referring to the formation of NECS means that the formation of the NECS as such is not forced or facilitated by any means apart from bringing the PSCs into single cell suspension. Spontaneous formation is opposite to an active aggregation of cells, which could be facilitated by clustering the cell together in a conical shaped well or in a droplet. As used herein, the “spontaneous” formation may also be referred to as “non-forced” formation or “passive” formation. Accordingly, in an embodiment, the NECS are allowed to form without forced aggregation. In a preferred embodiment, the NECS are allowed to form spontaneously in cell suspension. Without being bound by any particular theory, it is believed that the spontaneous formation of the NECS in part is due to proliferation of the PSCs and maybe in part due to spontaneous aggregation of one or more NECS.

In one embodiment, the tridimensional cell aggregates are allowed to spontaneously form NECS for further five days. In one embodiment, the tridimensional cell aggregates are allowed to spontaneously form NECS for at least further five days. In one embodiment, the tridimensional cell aggregates are allowed to spontaneously form NECS for further three to eight days. In one embodiment, the tridimensional cell aggregates are allowed to spontaneously form NECS for further three to ten days.

### Suspension Culture

In one embodiment the method further comprises the step of subjecting the suspension culture to agitation, to create a dynamic cell culture suspension. As used herein, the term “agitation” means providing movement of the cell culture medium for the purpose of maintaining a suspension culture. Agitation may be provided by any means suitable. In an embodiment, the suspension culture is subjected to agitation by shaking. In a further embodiment, the suspension culture is subjected to agitation at a speed of from about 5 rpms to about 80 rpms, preferably from about 20 rpms to about 70 rpms, more preferably from about 40 rpms to about 60 rpms. In one embodiment, the suspension culture is subjected to agitation at a speed of from about 30 rpms to about 100 rpms, preferably from about 40 rpms to about 90, more preferably 50 rpms to about 80 rpms. In one embodiment, the suspension culture is subjected to agitation at a speed of from about 5 rpms to about 80 rpms, preferably from about 20 rpms to about 70 rpms, more preferably from about 40 rpms to about 60 rpms. In one embodiment, the suspension culture is subjected to agitation at a speed of from about 50 rpms to about 80 rpms. In one embodiment, the suspension culture is subjected to agitation at a speed of from about 60 rpms to about 70 rpms. In a preferred embodiment, the suspension culture is subjected to agitation when starting to gradually reduce the concentration of the ROCK inhibitor. In an embodiment, the suspension culture is subjected to agitation from about day 0, day 1, day 2 or day 3, preferably the suspension culture is subjected to agitation from about day 1. In one embodiment, the suspension culture is subjected to agitation until the step of plating the NECS.

The method of the present invention comprises a step of differentiating the PSCs into NSCs. Allowing the PSCs to differentiate is not to be construed as a separate final step to be performed. One of ordinary skill in the art will appreciate that as used herein the terms “differentiate” and “differentiation” refer to the process wherein cells progress from an undifferentiated state to a differentiated state, from an immature state to a less immature state or from an immature state to a mature state, which occurs continuously as the method is performed and the cells are exposed to various factors facilitating the differentiation. This is for example but not limited to PSCs differentiating into NSCs. Changes in cell interaction and maturation occur as cells lose markers of undifferentiated cells or gain markers of differentiated cells. Loss or gain of a single marker can indicate that a cell has “matured or fully differentiated”.

In a preferred embodiment, the differentiation of the PSCs into NSCs is initiated at day 0. This implies that the differentiation of the PSCs into NSCs is initiated immediately after dissociating the PSCs into single cell suspension.

In an embodiment, the PSCs are contacted with an inhibitor of the TGFβR1/ALK5 receptor. As used herein, by the term “contacting” in reference to culturing cells is meant exposing the cells to e.g. a specific compound by bringing the specific compound in proximity to the cell in order to produce “contacted” cells. The contacting may be accomplished using any suitable means. A non-limiting example of contacting is by adding the compound to a cell culture medium of the cells. The contacting of the cells is assumed to occur as long as the cells and specific compound are in proximity, e.g. the compound is present in a suitable concentration in the cell culture medium. Referring to “contacting cells with X” may be regarded as synonymous with “culturing cells in a cell culture medium comprising X”. Furthermore, as used herein, the term “inhibitor” in reference to inhibiting a signaling target or a signaling target pathway refers to a compound that interferes with (i.e. reduces or eliminates or suppresses) a resulting target molecule or target compound or target process, such as a particular differentiation outcome, (for example, suppresses an active signaling pathway promoting a default cell type differentiation, thereby inducing differentiation into a non-default cell type) when compared to an untreated cell or a cell treated with a compound that does not inhibit a treated cell or tissue.

“OTX2” as used herein refers to Orthodenticle Homeobox 2 gene, transcript or protein, and it is a marker of anterior brain structures during embryonic development including the neural progenitor cells.

“PAX6” as used herein refers to “Paired Box 6” gene, transcript or protein and it is a marker of anterior brain structures during embryonic development including the neural progenitor cells.

“SOX2” as used herein refers to “SRY-Box Transcription Factor 2” gene, transcript or protein and it is a marker of neural progenitor cells.

“FOXG1” as used herein refers to “Forkhead Box G1” gene, transcript or protein and it is a marker of forebrain cells during embryonic development.

“ZO1” or “TJP1” as used herein refers to “Tight Junction Protein 1” gene, transcript or protein acts as a tight junction adaptor protein. It is used to define the apical part, lumen, of neural rosettes.

In one embodiment, the present invention relates to a method for obtaining neural stem cells, wherein at least 80% of the cells co-express PAX6 and OTX2.

In one embodiment, the present invention relates to a method for obtaining neural stem cells, wherein at least 85% of the cells co-express PAX6 and OTX2.

In one embodiment, the present invention relates to a method for obtaining neural stem cells, wherein at least 90% of the cells co-express PAX6 and OTX2.

In one embodiment, the present invention relates to a method for obtaining neural stem cells, wherein about 93%% of the cells co-express PAX6 and OTX2.

In one embodiment, the present invention relates to a method for obtaining neural stem cells, wherein at least 80% of the cells co-express PAX6, SOX2 and OTX2.

In one embodiment, the present invention relates to a method for obtaining neural stem cells, wherein at least 85% of the cells co-express PAX6, SOX2 and OTX2.

In one embodiment, the present invention relates to a method for obtaining neural stem cells, wherein at least 90% of the cells co-express PAX6, SOX2 and OTX2.

In one embodiment, the present invention relates to a method for obtaining neural stem cells, wherein about 93%% of the cells co-express PAX6, SOX2 and OTX2.

### Inhibitor of the Small Mothers Against Decapentaplegic (SMAD) Protein Signaling Pathway

As used herein “inhibitor of the Small Mothers Against Decapentaplegic (SMAD) protein signaling pathway” refers to a compound that specifically inhibits the Small Mothers Against Decapentaplegic (SMAD) protein signaling pathway. Examples of inhibitor of Small Mothers Against Decapentaplegic (SMAD) protein signaling may be selected from the group comprising GW788388, LDN-193189, LY2157299, LY364947, NOGGIN, RepSox, SB431542, and TEW-7197.

As used herein, “GW788388” denotes a small molecule chemical name N-(oxan-4-yl)-4-[4-(5-pyridin-2-yl-1H-pyrazol-4-yl)pyridin-2-yl]benzamide and CAS no: 452342-67-5.

As used herein, “LDN-193189” denotes a compound with the IUPAC name 44644-(Piperazin-1-yl)phenyl)pyrazolo[1,5-a]pyrimidin-3-yl)quinoline and CAS no: 1062368-24-4.

As used herein, “LY2157299” denotes a small molecule, which is potent TGFβ receptor I (TGFβRI) inhibitor with alternative name Galunisertib and chemical name 4-[2-(6-methylpyridin-2-yl)-5,6-dihydro-4H-pyrrolo[1,2-b]pyrazol-3-yl]quinoline-6-carboxamide, and CAS no: 700874-72-2.

As used herein, “LY364947” denotes compound with the IUPAC name 4-[3-(2-pyridinyl)-1H-pyrazol-4-yl]-quinoline and CAS no: 396129-53-6.

As used herein, “NOGGIN” denotes a secreted homodimeric glycoprotein that binds to and inactivates members of the transforming growth factor-beta (TGF-β) superfamily of signaling proteins, such as bone morphogenetic protein-4 (B MP 4). NOGGIN is typically a 65 kDa protein expressed in human cells as a glycosylated, disulfide-linked dimer.

As used herein, “RepSox” denotes a small molecule, which is a potent and selective inhibitor of TGF-βR1 with alternative names E-616452, SJN 2511, ALK5 Inhibitor II, and chemical name 2-(3-(6-Methylpyridine-2-yl)-1H-pyrazol-4-yl)-1,5-naphthyridine, and CAS no: 446859-33-2.

As used herein, “SB431542” denotes a compound with the chemical name 4-[4-(1,3-benzodioxol-5-yl)-5-(2-pyridinyl)-1H-imidazol-2-yl]benzamide and CAS no: 301836-41-9.

As used herein, “TEW-7197” denotes a small molecule with alternative name Vactosertib and chemical name 2-fluoro-N[[5-(6-methylpyridin-2-yl)-4-([1,2,4]triazolo[1,5-a]pyridin-6-yl)-1H-imidazol-2-yl]methyl]aniline and CAS no: 1352608-82-2.

method for obtaining in vitro neural stem cell (NSC) lines from pluripotent stem cells (PSCs).

In one embodiment, the PSCs are contacted with a single SMAD inhibitor or cultured in a medium comprising a single SMAD inhibitor. In one embodiment, the PSCs are contacted with RepSox or cultured in a medium comprising RepSox. In one embodiment, the PSCs are contacted with GW788388 or cultured in a medium comprising GW788388.

In one embodiment, the concentration of RepSox is from about 1 μM to about 200 μM, preferably from about 10 μM to about 100 μM, more preferably from about 20 μM to about 80 μM, more preferably from about 30 μM to about 70 μM, more preferably from about 40 μM to about 60 μM, even more preferably from about 45 μM to about 55 μM.

In a particular embodiment, the PSCs are contacted with RepSox in a concentration of about 50 μM.

In an embodiment, the PSCs are contacted with RepSox from day 0. By contacting the PSCs with RepSox at day 0 to differentiation of the cells starts as the cells are brought into suspension. Accordingly, the cells allowed to form NECS are already undergoing the early phase of differentiation towards becoming NSCs. In a more specific embodiment, the PSCs are contacted with RepSox from day 0 to the step of expanding the NSCs.

In an embodiment, the PSCs are contacted with GW788388 from day 0. By contacting the PSCs with GW788388 at day 0 to differentiation of the cells starts as the cells are brought into suspension. Accordingly, the cells allowed to form NECS are already undergoing the early phase of differentiation towards becoming NSCs. In a more specific embodiment, the PSCs are contacted with GW788388 from day 0 to the step of expanding the NSCs.

In one embodiment, the present invention relates to a method for obtaining neural stem cells, wherein said method comprises a single SMAD inhibitor to produce NSC with more than 80% of the cells being OTX2/PAX6/SOX2 positive without the need of manual picking and isolation.

In another embodiment, the pluripotent stem cells are contacted with an inhibitor of SMAD protein signaling is RepSox or GW788388 to produce NSC with more than 80% of the cells being OTX2/PAX6/SOX2 without the need of manual picking and isolation.

The inventors identified these inhibitors of SMAD protein signaling as providing a population of NSCs which are more homogeneous, being more than 80% of the cells being OTX2/PAX6/SOX2 without the need of manual picking and isolation.

The inventors identified these inhibitors of SMAD protein signaling as providing a population of NSCs which are more homogeneous or highly pure, being more than 80% of the cells being OTX2/PAX6 or PAX6/SOX2 double positive. The inventors identified these inhibitors of SMAD protein signaling as providing a population of NSCs which are more homogeneous or highly pure, being more than 80% of the cells being OTX2/PAX6 or PAX6/SOX2 double positive, without the need of manual picking and isolation.

The inventors surprisingly found that single inhibition of SMAD protein signaling provides a population of NSCs which are suitable for upscaling.

In one embodiment, the present invention provides a method for upscaling, for example, but not limited, to flasks, tanks and/or bioreactors.

In another embodiment, the single inhibitor of SMAD protein signaling is RepSox in a concentration of from about 0.25 μM to about 200 μM, preferably from about 10 μM to about 150 μM, more preferably from about 15 μM to about 100 μM, even more preferably from about 20 μM to about 75 μM.

In another embodiment, the single inhibitor of SMAD protein signaling is GW788388 in a concentration of from about 0.1 ng/ml to about 150 ng/ml, preferably from about 10 ng/ml to about 90 ng/ml, more preferably from about 20 ng/ml to about 80 ng/ml, even more preferably from about 40 ng/ml to about 75 ng/ml.

In an optional step according to the method, the NSCs of the spontaneously formed NECS may be dissociated prior to plating onto a substrate. However, this step is not required as the NECS can be plated directly. Accordingly, in the following description of plating reference to plating NECS may equally apply if the optional step of dissociating the NECS has been performed, mutatis mutandis.

The method comprises a step of plating the NSCs in suspension on a substrate. By the term “plating” is meant distributing the NECS onto a suitable substrate. A person skilled in the art will know the appropriate technique for transfer of NECS comprising stem cells onto a substrate. Culturing the NECS on a substrate may also be referred to as two-dimensional culturing. The transition from suspension culture to a two-dimensional culture implies a continuous culturing of the cells to maintain viable conditions.

As used herein, the term “substrate” is to be understood as a surface allowing the growth of stem cells and onto which a coating may be provided. This may be but is not limited to well plates and beads. A person skilled in the art will readily acknowledge suitable substrates for culturing the cells and these are commercially available. Typical substrates include but are not limited to cell culture treated multi-well plates, such as The Scientific™ Nunc™ Cell-Culture Treated multi-well plates. According an embodiment of present invention, the NECS are plated onto a substrate coated with an extracellular matrix.

By the term “extracellular matrix” is meant extracellular molecules that are responsible for interactions with cell surface receptors, thus regulating cell behavior such as adhesion, proliferation, migration and differentiation, or serve a mechanical supportive function. In one embodiment, the coating on the coated substrate comprises laminin and/or fibronectin and/or vitronectin and/or collagen.

In one embodiment the NSCs of the present invention are used to prepare extracellular vesicles.

As used herein, the term “laminin” in reference to coating on plates refers a heterotrimeric molecule consisting of three subunits termed alpha, beta and gamma chains. The references herein are made to human laminin. Five kinds of a chains (alpha 1 to alpha 5), three kinds of beta chains (beta 1 to beta 3) and three kinds of gamma chains (gamma1 to gamma3) are known, and various combinations of these chains give rise to at least 12 kinds of laminin isoforms. For example, “laminin a5 beta1 gamma1” is herein referred to as “laminin-511”. The same will apply to other isoforms. By “fragment thereof” when referring to laminin is meant part of the intact laminin. For instance, it has been found that the E8 fragment of laminin-511 strongly adhere to human embryonic stem cells. Laminins and fragments thereof are commercially available from companies such as Biolamina AB or Nippi Inc.

As used herein, the term “fibronectin” in reference to coating on plates refers to a high-molecular weight (˜440 kDa) glycoprotein of the extracellular matrix that binds to membrane-spanning receptor proteins called integrins Similar to integrins, fibronectin binds extracellular matrix components such as collagen, fibrin, and heparan sulfate proteoglycans (e.g. syndecans).

As used herein, the term “vitronectin” in reference to coating on plates refers to a glycoprotein of the hemopexin family which is abundantly found in serum, the extracellular matrix and bone.

As used herein, the term “collagen” in reference to coating on plates refers to a structural protein in the extracellular space in the various connective tissues in animal bodies.

As the main component of connective tissue, it is the most abundant protein in mammals making 25% to 35% of the whole-body protein content. Collagen consists of amino acids wound together to form triple-helices to form of elongated fibrils.

In a preferred embodiment, the extracellular matrix comprises a laminin or a fragment thereof, preferably selected from the group consisting of laminin-511 and laminin-521. In another embodiment, the laminin or fragment thereof is a combination of laminin-511 and laminin-521. In one embodiment the matrix comprises laminin-511 and/or laminin-521 and one or more further laminin(s). In one embodiment, the laminin is an intact laminin protein. In another embodiment, the laminin is a fragment of the intact laminin protein. In a further embodiment, the concentration of the laminin is from about 0.001 μg/cm² to about 50 μg/cm², preferably from about 0.1 μg/cm² to about 25 μg/cm², more preferably from about 0.1 μg/cm² to 10 μg/cm², more preferably from about 0.1 μg/cm² to about 5, more preferably from about 0.25 μg/cm² to about 1 μg/cm², even more preferably about 0.5 μg/cm².

In an embodiment, at least 90% of the NECS have a diameter of less than 500 μm prior to the step of plating the NECS containing NSCs, preferably the NECS have a diameter of less than 500 μm prior to the step of plating the NECS containing NSCs. In an embodiment, at least 50%, 60%, 70%, 80%, 90%, 95%, 96%, 97%, 98%, or 99% of the NECS have a diameter of less than 400 μm prior to the step of plating the NECS containing NSCs, preferably at least 80% of the NECS have a diameter of less than 400 μm prior to the step of plating the NECS containing NSCs, more preferably at least 90% of the NECS have a diameter of less than 400 μm prior to the step of plating the NECS containing NSCs. In an embodiment, at least 50%, 60%, 70%, 80%, 90% of the NECS have a diameter of less than 300 μm prior to the step of plating the NECS containing NSCs, preferably at least 80% of the NECS have a diameter of less than 300 μm prior to the step of plating the NECS containing NSCs. In a preferred embodiment, at least 90% of the NECS have a diameter of less than 500 μm prior to the step of plating the NECS containing NSCs, preferably the NECS have a diameter of less than 500 μm prior to the step of plating the NECS containing NSCs.

In a further embodiment, the NECS are plated when at least 50%, 60%, 70%, 80%, 90%, 95%, or 99% of the NECS containing NSCs express the marker SOX2.

### Neural Rosette

As used herein the term “neural rosette” refers to a self-forming organization of neuroectodermal cells and NSCs in which a cluster of neuroectodermal cells and NSCs with a circle-like morphology expand radially from the center. Without being bound by any particular theory, forming neural rosettes is an inherent developmental property of NSCs in culture, and are easily visualized in two-dimensional culture. Neural rosettes might be present in the NECS before plating into two-dimensional culture, although more difficult to visualize. According to the present invention, the radial arrangement of the NSCs is substantially formed by the cells themselves, with the proviso of providing the neuroectodermal cells viable conditions as well as providing factors necessary for maintaining and furthering the cells along the neural lineage. A person skilled in the art will recognize that neural rosette formation may undertake already in the NECS in suspension culture. However, the neural rosette formation is readily observable once the allowed to form on a two-dimensional substrate.

The method comprises a step of maintaining and expanding the NSCs to establish the neural stem cell (NSC) lines. As used herein the term “maintaining” and “maintenance” may be used interchangeably and refer to providing the culture conditions that keep the cells viable and proliferative. As used herein, the terms “expanding” and “expansion” may be used interchangeably and refer to the proliferation of the cell population, i.e. the NSCs are provided with conditions that allow them to continuously grow and divide. In an embodiment, the step of expanding the NSCs comprises the further steps of dissociating the NSCs into single cell suspension, contacting the NSCs with a ROCK inhibitor, replating the NSCs in single cell suspension on a second substrate, and allowing the NSCs to reform neural rosettes. In a further embodiment, the steps are repeated for maintaining and expanding the neural stem cell (NSC) line. In an embodiment, the NSCs are dissociated into single cell suspension by contacting the NSCs with a cell dissociation agent, such as trypsin or TrypLE Select. In an embodiment, the concentration of the ROCK inhibitor is about 0.5 μM to about 50 μM, preferably from about 5 μM to about 25 μM. In an embodiment, the ROCK inhibitor is Y-27632.

In an embodiment of the method, the PSCs are allowed to differentiate for about 5 days to about 15 days, preferably for about 7 days to about 13 days, more preferably for about 9 days to about 11 days, even for preferably for about 10 days. Furthermore, in an embodiment, the NSCs are plated after about 4 days to after about 15 days, preferably after about 5 days to after about 10 days, more preferably after about 5 days to after about 8 days, even more preferably after about 6 days. In a further embodiment, the step of maintaining and expanding the NSCs is initiated from about 1 day to about 8 days after plating the NSCs, preferably about 3 days to about 5 days after plating the NSCs, more preferably about 4 days after plating the NSCs.

In a preferred embodiment, the method does not comprise a step of isolating neural rosettes. As used herein, the term “isolating neural rosettes” means identifying neural rosettes that have formed properly and are believed viable for further expansion. Such identification may be facilitated by analysis of certain markers and isolation may be facilitated by manual picking under the microscope. However, the present method does not require a separate step of isolating neural rosettes. Accordingly, in a preferred embodiment the method does not comprise a step of manually selecting neural rosettes for maintaining and expanding the NSCs. It follows substantially all the formed neural rosettes are maintainable and expandable, and in a preferred embodiment, substantially all the formed neural rosettes are maintained and expanded. By “substantially all” is meant that some neural rosettes may not be viable and thus not maintainable and expandable.

By the term “highly pure” neural stem cell population, it is meant a homogeneous NSC population with more than 80% of the cells being OTX2/PAX6 double positive or PAX6/SOX2 double positive, without the need of manual picking and isolation.

In one embodiment, the NSCs obtained by the methods of the present invention are 80% double positive for OTX2/PAX6 or PAX6/SOX2.

In one embodiment, the NSCs obtained by the methods of the present invention are 80% double positive for OTX2/PAX6 or PAX6/SOX2, without the need of manual picking and isolation.

In one embodiment, the NSCs obtained by the methods of the present invention are at least 80% double positive for OTX2/PAX6 or PAX6/SOX2.

In one embodiment, the NSCs obtained by the methods of the present invention are at least 80% triple positive for OTX2/PAX6/SOX2.

In one embodiment, the NSCs obtained by the methods of the present invention are at least 80% quadruple positive for OTX2/PAX6/SOX2/FOXG1.

Another aspect of the present invention relates to a method for inducing neuroectodermal cells from PSCs, comprising the steps of contacting the PSCs with RepSox, and allowing the PSCs to differentiate into neuroectodermal cells. In an embodiment, the neuroectodermal cells are NSCs. In a preferred embodiment, the concentration of RepSox is higher than about 10 μM, 20 μM, 30 μM, 40 μM, or 45 μM, preferably higher than about 20 μM. Furthermore, in a preferred embodiment, the concentration of RepSox is less than about 200 μM, 150 μM, 100 μM, 80 μM, or 60 μM, preferably less than about 70 μM. In an embodiment, the concentration of RepSox is from about 1 μM to about 200 μM, preferably from about 10 μM to about 100 μM, more preferably from about 20 μM to about 80 μM, more preferably from about 30 μM to about 70 μM, more preferably from about 40 μM to about 60 μM, even more preferably from about 45 μM to about 55 μM. In one particular embodiment, the PSCs are contacted with RepSox in a concentration of about 50 μM.

Another aspect of the present invention relates to a method for inducing neuroectodermal cells from PSCs, comprising the steps of contacting the PSCs with GW788388, and allowing the PSCs to differentiate into neuroectodermal cells. In an embodiment, the neuroectodermal cells are NSCs. In a preferred embodiment, the concentration of GW788388 is higher than about 0.1 ng/ml, 0.5 ng/ml, 1 ng/ml, 3 ng/ml, or 5 ng/ml, preferably higher than about 10 ng/ml. Furthermore, in a preferred embodiment, the concentration of GW788388 is less than about 100 ng/ml, 80 ng/ml, 60 ng/ml, 40 ng/ml, or 20 ng/ml, preferably less than about 10 ng/ml. In an embodiment, the concentration of GW788388 is from about 0.1 ng/ml to about 150 ng/ml, preferably from about 10 ng/ml to about 90 ng/ml, more preferably from about 20 ng/ml to about 80 ng/ml, even more preferably from about 40 ng/ml to about 75 ng/ml. In one particular embodiment, the PSCs are contacted with GW788388 in a concentration of about 10 ng/ml.

In one embodiment, the PSCs are not contacted with an inhibitor of the bone morphogenetic protein (BMP) signaling pathway. Specifically, in one embodiment the PSCs are not contacted with Noggin.

A further embodiment of the present invention relates to the use of the NSC lines obtained according to the methods of the first aspect for producing exosomes. Accordingly, an aspect of the present invention relates to a method for producing exosomes from NSC lines obtained according to the method of any one of the preceding embodiments, comprising the steps of allowing the NSCs to produce exosomes, and isolating the exosomes. Furthermore, in an aspect is provided an exosome obtained according to the aforementioned method.

### Exosome

As used herein, the term “exosome” refers to small vesicles or nanoscale vesicles (from 30-150 nm in diameter) having a membrane structure secreted from various cell types. Typically, the cells produce exosomes as a small membrane-bound vesicle of endocytic origin that is then released into the extracellular environment following fusion of multivesicular bodies with the plasma membrane. An exosome may serve as a carrier of molecules between various locations in a body or biological system. The exosome may comprise molecules such as nucleic acids (e.g. DNA, mRNA, miRNA), proteins, and/or other biomolecules, which molecules can be present on the surface, membrane and/or interior of the exosome.

In one embodiment, the exosomes of the present invention are obtained from NSCs wherein at least 80% of the cells are double positive for OTX2/PAX6 or PAX6/SOX2.

In one embodiment, the exosomes of the present invention are obtained from NSCs wherein at least 80% of the cells are triple positive for OTX2/PAX6/SOX2.

In one embodiment, the exosomes of the present invention are obtained from NSCs wherein at least 80% of the cells are quadruple positive for OTX2/PAX6/SOX2/FOXG1.

A further aspect relates to the exosome according to the present invention for use as a medicament. In an embodiment, the exosome is for use in the treatment of a neurodegenerative disorder. In one embodiment the neurodegenerative disorder is stroke. In one embodiment the neurodegenerative disorder is traumatic brain injury (TBI). In one embodiment the neurodegenerative disorder is Alzheimer's disease.

In one embodiment the present invention relates to exosomes derived from NSC with at least 80% of the cells being triple positive for OTX2/PAX6/SOX2, for intra-venous injection, intranasal delivery or intrathecal administration.

In one embodiment the present invention relates to exosomes derived from NSC obtained by the methods of the present invention, for intra-venous injection, intranasal delivery, or intrathecal administration.

### Dynamic Cell Culture Suspension

Suspension culture bioreactors allow for large-scale expansion and differentiation of stem cells and/or their progeny in a controlled and reproducible culture system. These systems offer a homogeneous culture environment where conditions such as temperature, pH, and oxygen concentration can be monitored and controlled. Furthermore, these systems permit the production of large numbers of cells under consistent culture conditions, and with minimal culture variability.

In a further embodiment, the dynamic cell culture is subjected to agitation at a speed of from about 5 rpms to about 80 rpms, preferably from about 20 rpms to about 70 rpms, more preferably from about 40 rpms to about 60 rpms. In one embodiment, the suspension culture is subjected to agitation at a speed of from about 30 rpms to about 100 rpms, preferably from about 40 rpms to about 90, more preferably 50 rpms to about 80 rpms. In one embodiment, the suspension culture is subjected to agitation at a speed of from about 5 rpms to about 80 rpms, preferably from about 20 rpms to about 70 rpms, more preferably from about 40 rpms to about 60 rpms. In one embodiment, the suspension culture is subjected to agitation at a speed of from about 50 rpms to about 80 rpms. In one embodiment, the suspension culture is subjected to agitation at a speed of from about 60 rpms to about 70 rpms.

### Culture Medium/Composition

A solid, liquid or semi-solid designed to support the growth of microorganisms or cells. Different types of commercial media are used for growing different types of cells.

In another aspect of the present invention is provided a method of maintaining and expanding a NSC line comprising the steps of culturing NSCs on a substrate, allowing the NSCs to reform neural rosettes, dissociating the NSCs into single cell suspension, contacting the NSCs with a ROCK inhibitor, and replating the NSCs on a second substrate. By the term “second substrate” is meant that the substrate may be identical to the initial first substrate. The second substrate may have the same or a different coating such as extracellular matrix. In a preferred embodiment, in the step of allowing the NSCs to reform neural rosettes the NSCs are also allowed to reach confluency prior to the step of dissociating the NSCs into single cell suspension. By the term “confluency” is to be construed as a measure of the proliferation of the cells in the culture medium and basically refers to the coverage of the culture vessel. As used herein, 100% confluency means that e.g. a dish is substantially covered by cells. In an embodiment, the NSCs are allowed to reach at least 50%, 60%, 70%, 80%, 90%, 95%, 99%, or 100% confluency. Preferably, the NSCs are allowed to reach 100% confluency. As used herein, carrying out the steps according to the method of maintaining and expanding the NSC line is referred to as a “passage”. In an embodiment, the steps are repeated to maintain and expand the NSC line for at least 3 passages, preferably for at least 5 passages, more preferably at least 10 passages, even more preferred at least 15 passages. Typically, the first three passages are the most critical. Some may refer to the line as established only after three to four initial passages.

In a further embodiment, the steps are repeated to maintain and expand the NSC line. In an embodiment, the ROCK inhibitor is Y-27632. In an embodiment, the concentration of the ROCK inhibitor is from about 0.5 μM to about 50 μM, preferably from about 5 μM to about 25 μM, even more preferably about 10 μM.

In a final aspect is disclosed a method for obtaining in vitro neural stem cells from PSCs, comprising the steps of dissociating the PSCs into single cells or aggregates comprising less than about 50 cells, culturing the PSCs in a suspension culture, allowing the PSCs in suspension to spontaneously form NECS, and differentiating the PSCs into neural stem cells

In an embodiment thereof, the aggregates comprise less than 40 cells, preferably less than 30 cells, more preferably less than 20 cells, even more preferably less than 10 cells. In an embodiment, the NSCs may be plated or maintained in suspension culture, and/or further differentiated. A person skilled in the art will readily recognize established protocols for the further differentiation depending on the desired end-product.

### Particular Embodiments

The invention is further described by the following non-limiting embodiments:
1. A method for obtaining neuroectodermal cells from pluripotent stem cells (PSCs) comprising the steps of:
   contacting said PSCs with ROCKi and a single SMAD inhibitor in suspension culture,
   allowing said PSCs in suspension to spontaneously form tridimensional cell aggregates,
   allowing said tridimensional cell aggregates to differentiate into neuroectodermal spheres with a diameter of less than 500 μm in dynamic cell culture suspension,
   allowing said neuroectodermal spheres to form neural rosettes, wherein said neural rosettes comprise neuroectodermal cells.
2. The methods according to embodiment 1, wherein said neural rosettes are allowed to be maintained and expanded into neural stem cell (NSC) lines.
3. The methods according to any one of the preceding embodiments, wherein the single SMAD inhibitor is RepSox or GW788388.
4. The method according to any one of the preceding embodiments, wherein RepSox is in the concentration of from about 20 μM to about 60 μM or wherein GW788388 is in the concentration from about 0.1 ng/ml to about 20 ng/ml.
5. The method according to any one of the preceding embodiments, where said neuroectodermal cells are neural stem cells.
6. The method according to any one of the preceding embodiments, where said neural stem cells are at least 80% double positive for OTX2/PAX6.
The method according to any one of the preceding embodiments, where said neural stem cells are at least 80% double positive for PAX6/SOX2.
8. The method according to any one of the preceding embodiments, where said neural stem cells are at least 80% triple positive for OTX2/PAX6/SOX2.
9. The method according to any one of the preceding embodiments, where said neural stem cells are at least 80% quadruple positive for OTX2/PAX6/SOX2/FOXG1.
10. A method for obtaining an in vitro neural stem cells, comprising the steps of:
   dissociating the PSCs into single cells,
   contacting said PSCs with ROCKi and a single SMAD inhibitor in suspension culture,
   allowing the PSCs in suspension to spontaneously form tridimensional cell aggregates
   allowing said tridimensional cell aggregates to differentiate into neuroectodermal spheres with a diameter of less than 500 μm in dynamic cell culture suspension,
   plating the NECS-containing neuroectodermal cells on a substrate or optionally dissociating the NECS-containing NSCs,
   allowing the NSCs to form neural rosettes and maintaining and expanding the NSCs to establish the NSC lines, without the need of manual picking and isolation.
11. The methods according to the preceding embodiment, wherein the PSCs are dissociated into single cells at day 0.
12. The methods according to any one of the preceding embodiments, wherein the PSCs are dissociated into single cells by contacting the PSCs with a cell dissociation agent, such as trypsin and/or TrypLE Select.
13. The methods according to any one of the preceding embodiments, wherein the concentration of the ROCK inhibitor is from about 0.5 μM to about 50 μM, preferably from about 5 μM to about 25 μM, even more preferably about 10 μM.
14. The methods according to any one of embodiments 7 and 13, wherein the ROCK inhibitor is Y-27632.
15. The methods according to any one of the preceding embodiments, wherein the PSCs are contacted with the ROCK inhibitor for about one day after the step of dissociating the PSCs into single cell suspension at day 0 at a concentration of from about 0.5 μM to about 50 μM, preferably from about 5 μM to about 25 μM, even more preferably about 10 μM.
16. The methods according to any one of the preceding embodiments, wherein the concentration of the ROCK inhibitor is gradually reduced from about day 1.
17. The methods according to any one of the preceding embodiments, further comprising a step of subjecting the suspension culture to agitation.
18. The methods according to embodiment 17, wherein the suspension culture is subjected to agitation from about day 0, day 1, day 2 or day 3, preferably the suspension culture is subjected to agitation from about day 1.
19. The methods according to any one of the embodiments 17 and 18, wherein the suspension culture is subjected to agitation until the step of plating the NSCs.
20. The methods according to embodiments 16 and 17, wherein the suspension culture is subjected to agitation when starting to gradually reduce the concentration of the ROCK inhibitor.
21. The methods according to any one of embodiments 17 to 20, wherein the suspension culture is subjected to agitation by shaking.
22. The methods according to any one of embodiments 17 and 21, wherein the suspension culture is subjected to agitation at a speed of from about 5 rpms to about 80 rpms, preferably from about 20 rpms to about 70 rpms, more preferably from about 40 rpms to about 60 rpms.
23. The methods according to any one of the preceding embodiments, wherein the PSCs substantially form NECS via proliferation and spontaneous aggregation.
24. The methods according to any one of the preceding embodiments, wherein at least 90% of the NECS have a diameter of less than 500 μm prior to the step of plating the NSCs, preferably the NECS have a diameter of less than 500 μm prior to the step of plating the NSCs.
25. The methods according to any one of the preceding embodiments, wherein at least 50%, 60%, 70%, 80%, 90%, 95%, 96%, 97%, 98%, or 99% of the NECS have a diameter of less than 400 μm prior to the step of plating the NSCs, preferably at least 80% of the NECS have a diameter of less than 400 μm prior to the step of plating the NSCs, more preferably at least 90% of the NECS have a diameter of less than 400 μm prior to the step of plating the NSCs.
26. The methods according to any one of the preceding embodiments, wherein at least 50%, 60%, 70%, 80%, 90% of the NECS have a diameter of less than 300 μm prior to the step of plating the NSCs, preferably at least 80% of the NECS have a diameter of less than 300 μm prior to the step of plating the NSCs.
27. The methods according to any one of the preceding embodiments, wherein the differentiation of the PSCs into NSCs is initiated at day 0.
28. The methods according to any one of the preceding embodiments, wherein the differentiation of the PSCs into NSCs is initiated immediately after dissociating the PSCs into single cell suspension.
29. The methods according to any one of the preceding embodiments, wherein the PSCs are contacted with an inhibitor of the TGFβR1/ALK5 receptor, such as RepSox.
30. The methods according to any one of the preceding embodiments, wherein the PSCs are contacted with an inhibitor of the TGFβ. type 2 receptor/ALK5 receptor, such as GW788388.
31. The methods according to embodiment 29, wherein the concentration of RepSox is from about 1 μM to about 200 μM, preferably from about 10 μM to about 100 μM, more preferably from about 20 μM to about 80 μM, more preferably from about 30 μM to about 70 μM, more preferably from about 40 μM to about 60 μM, more preferably from about 45 μM to about 55 μM, even more preferably about 50 μM.
32. The methods according to any one of embodiments 29 to 31, wherein the PSCs are contacted with RepSox from day 0 to the step of expanding the NSCs.
33. The methods according to any one of the preceding embodiments, wherein the NSCs are plated on a substrate comprising an extracellular matrix.
34. The methods according to embodiment 33, wherein the extracellular matrix is selected from the group consisting of fibronectin, vitronectin, collagen, and laminin, or a combination thereof, and/or fragments thereof.
35. The methods according to embodiment 34, wherein the extracellular matrix is a laminin.
36. The methods according to embodiment 35, wherein the laminin is selected from the group consisting of laminin-521, laminin-511, or a combination thereof, or fragments thereof.
37. The methods according to any one of the preceding embodiments, wherein the PSCs are allowed to differentiate for about 5 to about 15 days, preferably for about 10 days.
38. The methods according to any one of the preceding embodiments, wherein the NSCs are plated when at least 50%, 60%, 70%, 80%, 90%, 95%, or 99% of the NSCs express the marker SOX2.
39. The methods according to any one of the preceding embodiments, where said neural stem cells are at least 80% double positive for OTX2/PAX6.
40. The methods according to any one of the preceding embodiments, where said neural stem cells are at least 80% double positive for PAX6/SOX2.
41. The methods according to any one of the preceding embodiments, where said neural stem cells are at least 80% triple positive for OTX2/PAX6/SOX2.
42. The methods according to any one of the preceding embodiments, where said neural stem cells are at least 80% quadruple positive for OTX2/PAX6/SOX2/FOXG1.
43. The methods according to any one of the preceding embodiments, wherein the NSCs are plated after about 4 days to after about 15 days, preferably after about 5 days to after about 10 days, more preferably after about 5 days to after about 8 days, even more preferably after about 6 days.
44. The methods according to any one of the preceding embodiments, wherein the step of maintaining and expanding the NSCs is initiated from about 3 days to about 8 days after plating the NSCs, preferably about 3 days to about 5 days after plating the NSCs, more preferably about 4 days after plating the NSCs.
45. The methods according to any one of the preceding embodiments, wherein the methods do not comprise a step of isolating neural rosettes.
46. The methods according to any one of the preceding embodiments, wherein the methods do not comprise a step of manually selecting neural rosettes for maintaining and expanding the NSCs.
47. The methods according to any one of the preceding embodiments, wherein substantially all formed neural rosettes are maintainable and expandable.
48. The methods according to any one of the preceding embodiments, wherein substantially all the formed neural rosettes are maintained and expanded.
49. The methods according to any one of the preceding embodiments, wherein the PSCs are cultured in a first cell culture medium from day 0 to day 1.
50. The methods according to embodiment 49, wherein the first cell culture medium is Nutristem.
51. The methods according to any one of embodiments 49 and 50, wherein the PSCs are cultured in a second cell culture medium from day 1.
52. The methods according to embodiment 51, wherein the second cell culture medium is DMEM/F12.
53. The methods according to any one of the preceding embodiments, wherein the cell culture medium from day 1 comprises N2 supplement in a concentration from about 0.1% (v/v) to about 5% (v/v), preferably from about 0.5% (v/v) to about 2.5% (v/v), more preferably about 1% (v/v).
54. The methods according to any one of the preceding embodiments, wherein the NSCs are maintained and expanded in a cell culture medium comprising B27 in a concentration from about 0.01% (v/v) to about 5% (v/v), preferably from about 0.5% (v/v) to about 2.5% (v/v), more preferably about 0.1% (v/v).
55. The methods according to any one of the preceding embodiments, wherein the step of expanding the NSCs comprises the further steps of:
   dissociating the plated NSCs into single cell suspension,
   contacting the NSCs with a ROCK inhibitor,
   replating the NSCs in single cell suspension on a second substrate, and
   allowing the NSCs to reform neural rosettes.
56. The methods according to embodiment 55, wherein the further steps are repeated for maintaining and expanding the neural stem cell (NSC) line.
57. The methods according to any one of embodiments 55 and 56, wherein the NSCs are dissociated into single cell suspension by contacting the NSCs with a cell dissociation agent, such as trypsin and/or TrypLE Select.
58. The methods according to any one of embodiments 55 to 57, wherein the concentration of the ROCK inhibitor is from about 0.5 μM to about 50 μM, preferably from about 5 μM to about 25 μM, even more preferably about 10 μM.
59. The methods according to any one of embodiments 55 to 58, wherein the ROCK inhibitor is Y-27632.
60. A method for obtaining neuroectodermal cells from pluripotent stem cells (PSCs), comprising the steps of:
   contacting the PSCs with RepSox, and
   allowing the PSCs to differentiate into neuroectodermal cells.
61. The method according to embodiment 60, wherein the neuroectodermal cells are neural stem cells (NSCs).
62. The method according to any one of embodiments 60 and 61, wherein the concentration of RepSox is higher than about 10 μM, 20 μM, 30 μM, 40 μM, preferably higher than about 40 μM.
63. The method according to any one of embodiments 60 to 62, wherein the concentration of RepSox is less than about 200 μM, 150 μM, 100 μM, 90, μM, 80 μM, 70 μM, 60 μM, preferably less than 60 μM.
64. The method according to any one of embodiments 60 to 63, wherein the concentration of RepSox is from about 1 μM to about 200 μM, preferably from about 10 μM to about 100 μM, more preferably from about 20 μM to about 80 μM, more preferably from about 30 μM to about 70 μM, more preferably from about 40 μM to about 60 μM, more preferably from about 45 μM to about 55 μM, even more preferably about 50 μM.
65. The method according to any one of embodiments 60 to 64, wherein the PSCs are not contacted with a BMP inhibitor.
66. The method according to any one of embodiments 60 to 64, wherein the PSCs are not contacted with Noggin.
67. The method according to any one of embodiments 60 to 65, wherein the PSCs are not contacted with an inhibitor of the bone morphogenetic protein (BMP) signaling pathway.
68. Use of the neural stem cell (NSC) lines obtained according to the methods of any one of the preceding embodiments, for producing extracellular vesicles.
69. A method for producing exosomes from neural stem cell (NSC) lines obtained according to the methods of any one of the embodiments 1 to 59, comprising the steps of:
   allowing the NSCs to produce extracellular vesicles, and
   isolating the extracellular vesicles.
      Use of the neural stem cell (NSC) lines obtained according to the methods of any one of the preceding embodiments, for producing exosomes.
70. A method for producing exosomes from neural stem cell (NSC) lines obtained according to the methods of any one of the embodiments 1 to 59, comprising the steps of:
   allowing the NSCs to produce exosomes, and
   isolating the exosomes.
71. An exosome obtained according to the method of embodiments 0.
72. The exosome according to embodiment 71, for use as a medicament.
73. The exosome according to embodiment 72, for use in the treatment of a neurodegenerative disorder.
74. The exosome according to embodiment 73, wherein the neurodegenerative disorder is selected from stroke, traumatic brain injury (TBI), and Alzheimer's disease.
75. A method for maintaining and expanding a neural stem cell (NSC) line comprising the steps of:
   culturing NSCs on a substrate,
   allowing the NSCs to reform neural rosettes,
   dissociating the NSCs into single cell suspension,
   contacting the NSCs with a ROCK inhibitor, and
   replating the NSCs on a second substrate.
76. The method according to embodiment 75, wherein the steps are repeated to maintain and expand the neural stem cell (NSC) line for at least 5 passages, preferably for at least 10 passages.
77. The method according to any one of embodiments 75 and 76, wherein NSCs are allowed to reach confluency prior to the step of dissociating the NSCs into single cell suspension.
78. The method according to embodiment 77, the NSCs are allowed to reach at least 50%, 60%, 70%, 80%, 90%, 95%, 99%, or 100% confluency, preferably at least 90% confluency.
79. The method according to any one of embodiments 75 to 78, wherein the concentration of the ROCK inhibitor is from about 0.5 μM to about 50 μM, preferably from about 5 μM to about 25 μM, even more preferably about 10 μM.
80. The method according to any one of embodiments 75 to 79, wherein the ROCK inhibitor is Y-27632.
81. A method for obtaining in vitro neural stem cells from pluripotent stem cells (PSCs), comprising the steps of:
   dissociating the PSCs into single cells or aggregates comprising less than about 50 cells,
   culturing the PSCs in a suspension culture,
   allowing the PSCs in suspension to spontaneously form (NECS), and
   differentiating the PSCs into neural stem cells.
82. The method according to embodiment 81, wherein the aggregates comprise less than 40 cells, preferably less than 30 cells, more preferably less than 20 cells, even more preferably less than 10 cells.
83. The method according to any one of embodiments 81 and 82, wherein the NSCs may be plated or maintained in suspension culture, and/or further differentiated.
84. A highly pure population of neuroectoderm cells obtained by the methods according to any one of embodiments 1-67.
85. The highly pure population of neuroectoderm cells of embodiment 84, for further differentiation into neural or glia cells, for use as a medicament.
86. The neural stem cells or glia cells of embodiment 85, for use in the treatment of neurodegenerative disorders by transplanting said neural or glia cells or tissue or organ derived from stem cells into a subject in need thereof.
87. A kit for regenerating and/or repairing and/or building a tissue or an organ, wherein said kit comprises:
   i. a device
   ii. a population of neuroectoderm cells according to embodiments 10-12, for further differentiation into neural cells or glia cells, or an exosome or exosome population of embodiments 7-9,
   iii. a biocompatible scaffold or matrix,
   iv. at least one growth factor or functional fragment thereof;
   v. an agent selected from the group consisting of a ROCK inhibitor and/or a single SMAD inhibitor, such as RepSox or GW788388; and

optionally, directions for preparing, maintaining and/or using the cells including any cell culture or tissue or organ derived therefrom.

The invention is even further described by the following non-limiting embodiments:
89. A method for obtaining in vitro neural stem cell (NSC) lines from pluripotent stem cells (PSCs), comprising the steps of:
   dissociating the PSCs into single cells,
   culturing the PSCs in a suspension culture,
   allowing the PSCs in suspension to spontaneously form (NECS),
   differentiating the PSCs into NSCs,
   optionally dissociating the NSCs,
   plating the NSCs on a substrate,
   allowing the NSCs to form neural rosettes, and
   maintaining and expanding the NSCs to establish the NSC lines.
90. The method according to embodiment 89, wherein the PSCs are contacted with a ROCK inhibitor in the step of bringing the PSCs into single cell suspension.
91. The method according to embodiments 89-90, further comprising the step of subjecting the suspension culture to agitation.
92. The method according to embodiment 91, wherein the suspension culture is subjected to agitation from about day 1.
93. The method according to embodiments 89-92, wherein the PSCs form NECS by proliferation and/or non-forced aggregation.
94. The method according to embodiments 89-92, wherein the NECS have a diameter of less than 500 μm prior to the step of plating the NSCs.
95. The method according to embodiments 89-94, wherein the PSCs are contacted with RepSox in a concentration from about 1 μM to about 100 μM.
96. The method according to embodiments 89-95, wherein the step of expanding the NSCs comprises the further steps of:
   dissociating the plated NSCs into single cell suspension,
   contacting the NSCs with a ROCK inhibitor,
   replating the NSCs in single cell suspension on a second substrate, and
   allowing the NSCs to reform neural rosettes.
97. The method according to embodiment 96, wherein the further steps of the expanding the NSCs are repeated to maintain and expand the neural stem cell (NSC) line for at least 10 passages.
98. Use of the neural stem cell (NSC) lines obtained according to the method of any one of embodiments 89-97, for producing extra cellular vesicles.
99. Use of the neural stem cell (NSC) lines obtained according to the method of any one of embodiments 89-97, for producing exosomes.
100. A method for obtaining neuroectodermal cells from pluripotent stem cells (PSCs), comprising the steps of:
   contacting the PSCs with RepSox, and
   allowing the PSCs to differentiate into neuroectodermal cells, wherein the concentration of RepSox is from about 40 μM to about 60 μM.
101. The method according to embodiment 100, wherein the neuroectodermal cells are neural stem cells (NSCs).
102. The method according to any one of embodiments 100-101, wherein the PSCs are not contacted with Noggin.
103. A method of maintaining and expanding a neural stem cell (NSC) line comprising the steps of:
   culturing NSCs on a substrate,
   allowing the NSCs to reform neural rosettes,
   bringing the NSCs into single suspension,
   contacting the NSCs with a ROCK inhibitor, and
   replating the NSCs on a second substrate,

wherein the steps are repeated for at least 10 passages.
104. The method according to any one of embodiments 90 to 95 and 103, wherein the concentration of the ROCK inhibitor is from about 0.5 μM to about 50 μM.

### EXAMPLES

The following are non-limiting examples of protocols for carrying out the invention.

### List of Abbreviations

Cycle threshold (CT) values;

### DMEM/F12 (Dulbecco's Modified Eagle Medium/Ham's F-12 Medium)

Good manufacturing practice (GMP);
Good tissue practices (GTP);
Human embryonic stem cells (hESC);
Human induced pluripotent stem cell (hiPSC);
Human pluripotent stem cells (hPSCs);
Human recombinant laminin (hrLN)

### Laminin (LN);

Neural stem cells (NSCs)
Neuroectodermal spheres (NECS)

### Orthodenticle Homeobox 2 (OTX2);

### Paired Box 6 (PAX6);

Pluripotent stem cells (PSCs)
Real-time polymerase chain reaction (PCR);
Rho-associated coiled-coil containing kinases (ROCK);
Inhibitor of Rho-associated coiled-coil containing kinases (ROCKi);

### Small Mothers Against Decapentaplegic (SMAD);

General Methods of Preparation

Culture of hESCs

An internally generated hESC line was maintained on human recombinant laminin (hrLN) coated plates (Biolaminin 521 LN, Biolamina) in NutriStem hPSC XF medium (Biological Industries), in a 5% CO₂ incubator at 37° C. and passaged enzymatically at 1:10-1:20 ratio every 3-5 days. For passaging, confluent cultures were washed once with phosphate buffered saline (PBS) without calcium and magnesium ions and incubated for 5 min at 37° C. with TrypLE Select (GIBCO, Thermo Fisher Scientific). The enzyme was then carefully removed and the cells were collected in fresh NutriStem hPSC XF medium by gentle pipetting to obtain single cell suspension and the required volume plated on a freshly hrLN-521 coated dish. After passage, the medium was replaced with fresh prewarmed NutriStem hPSC XF medium and changed daily.

### Example 1: Differentiation Protocol for NECS and Rosette Formation

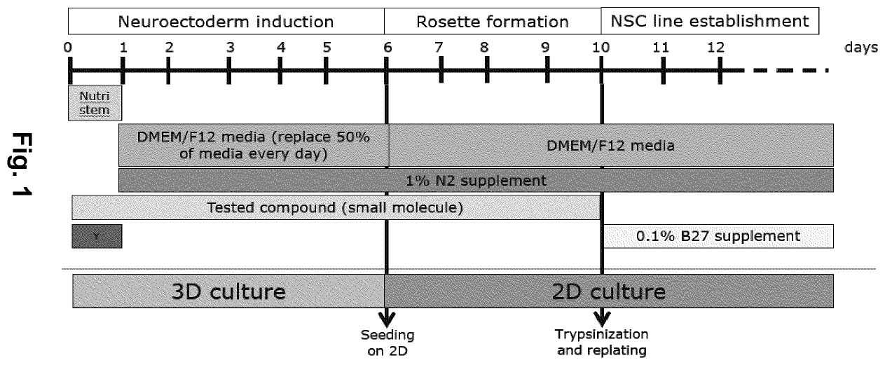

The experimental protocol used for neural induction of hESC and subsequent generation and NSC line establishment can roughly be divided in three main stages—Neuroectoderm induction and NECS formation; NECS plating and rosette formation and finally NSC line establishment by replating the cells in expansion media. The protocol is schematically presented in FIG. 1.

50-90% confluent hESC were washed with PBS^{-/-} and dissociated from monolayer culture with TrypLE™ Select (Gibco) at 37° C. for approximately 5 minutes to obtain single-cell suspension. The single-cell suspension of hESCs was resuspended in NutriStem® supplemented with 10 μM ROCKi (Y-27632) and as standard 1 mL with cell densities of 1×10⁵-2×10⁵ cells per mL was seeded into one single well (around 3.5 cm²) of a 12-well plates (Day 0).

At day 1, around 50% of the medium was exchanged with NECS media consisting of DMEM/F12 GlutaMAX™ supplement (Gibco™) supplemented with 20 U/mL Penicillin-streptomycin, 1% N-2 Supplement (Gibco™) and GW788388 or RepSox in different concentrations, without ROCKi. To initiate rotary NECS formation, plates were from day 1-6 placed on a Multi Bio 3D Mini-shaker (BioSan) at different speeds, timeslots, levels and angle. As an example, one optimal condition was:

Speed: 40-60 rpm

Angle (turning angle): 240-360 degrees, 10-15 sec.

Vibration (rocking angle): 5 degrees, 3-5 sec.

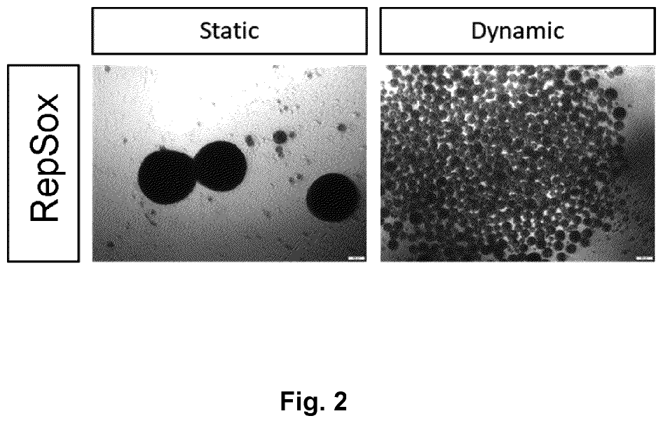

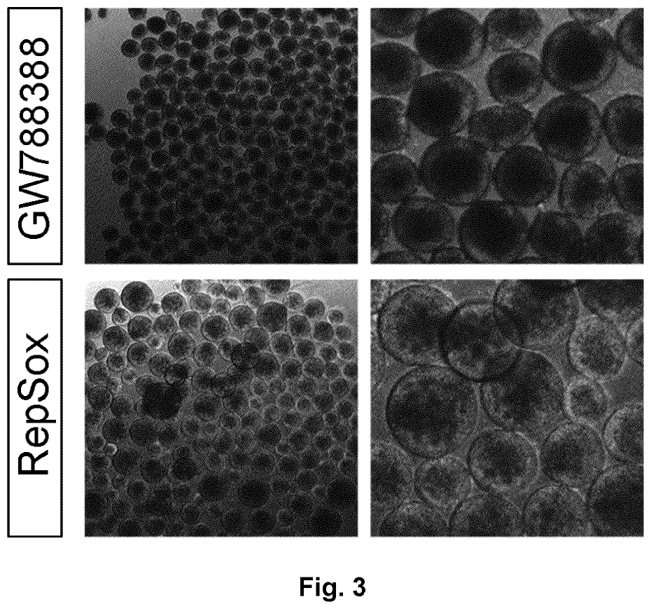

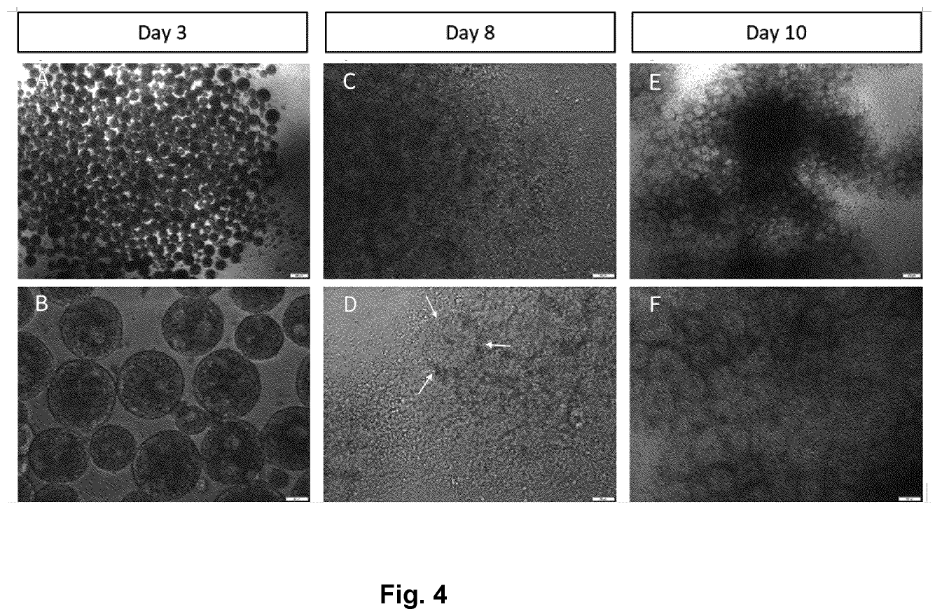

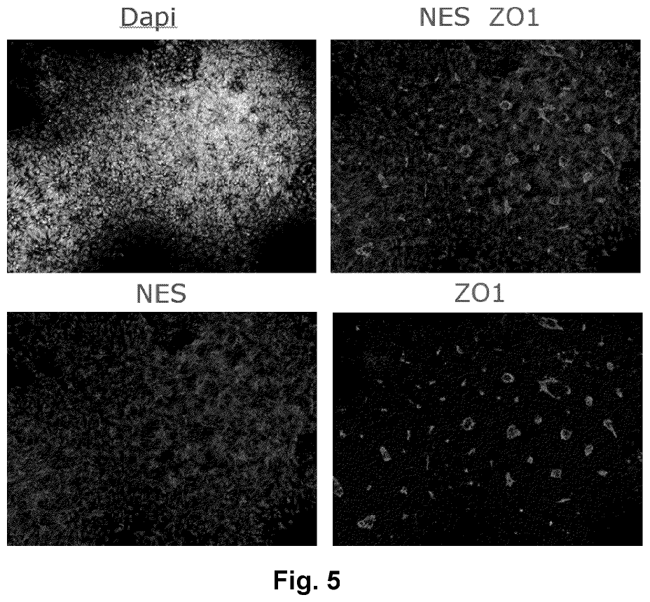

The NECS media was exchanged everyday by removing half of the media and adding fresh medium. The comparison between static versus dynamic culture is shown in FIG. 2, showing small and uniform structures formed by the dynamic conditions. Compared between RepSox and GW788388 conditions, the treatment with RepSox produced more transparent NECS, indicating less dense tridimensional structures (FIG. 3). After 6 days in dynamic conditions all NECS were collected and plated in 2D on 48-well plates, laminin coated. For coating, 1:50 natural mouse laminin (L2020-1MG, Sigma-Aldrich, 1 mg/ml) was performing fine, but the type of laminin can be also extended to LN-521 or LN-511 (BioLamina). From day 7 to day 10, now in attachment in 2D, all media was removed, and cells were re-feed with fresh NECS media. An example of the different steps can be shown in FIG. 4, with images after Day 3 (dynamic suspension), Day 8 (neural rosettes in 2D) and Day 10 (before single cell dissociation). Neural rosettes were clearly visible by day 10 in RepSox condition (See FIG. 5), and cells were positive for NES and each neural rosette showed a positive ZO1 lumen.

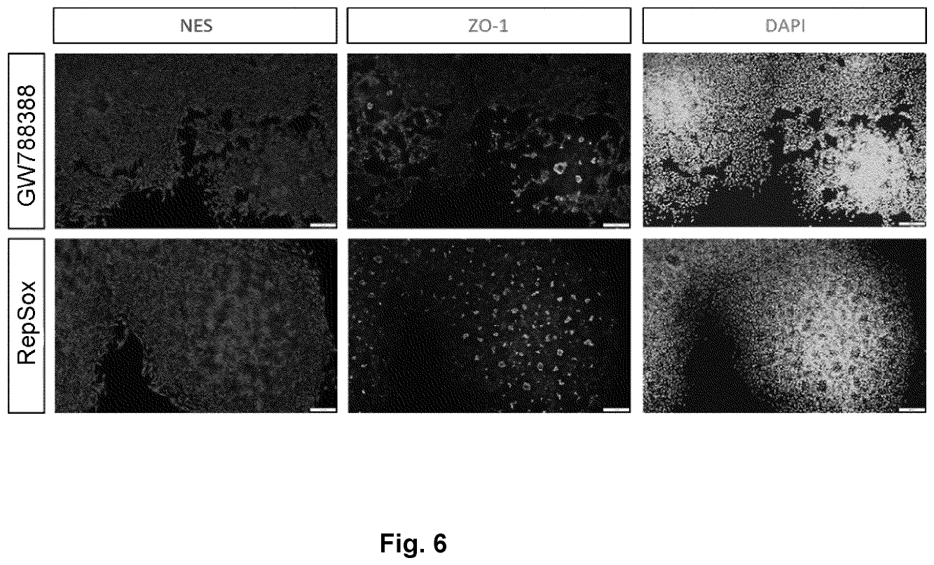

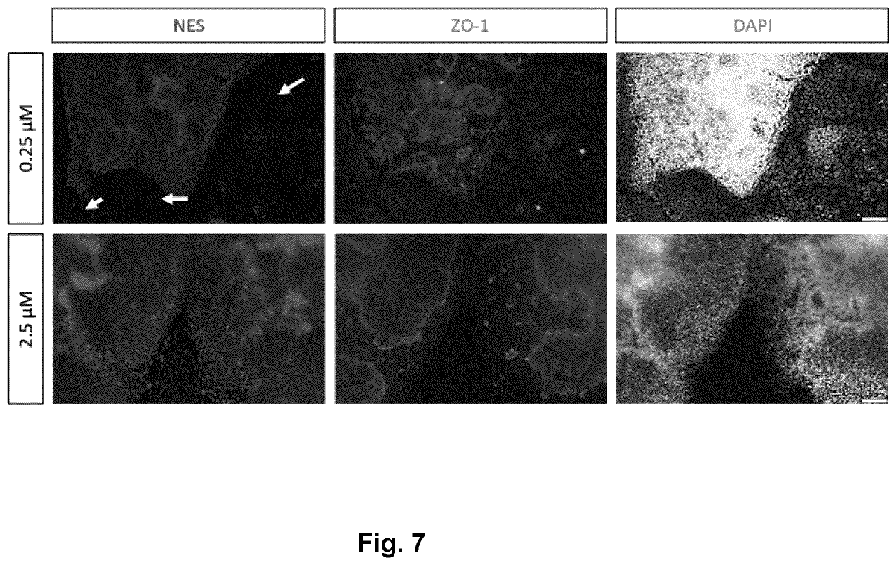

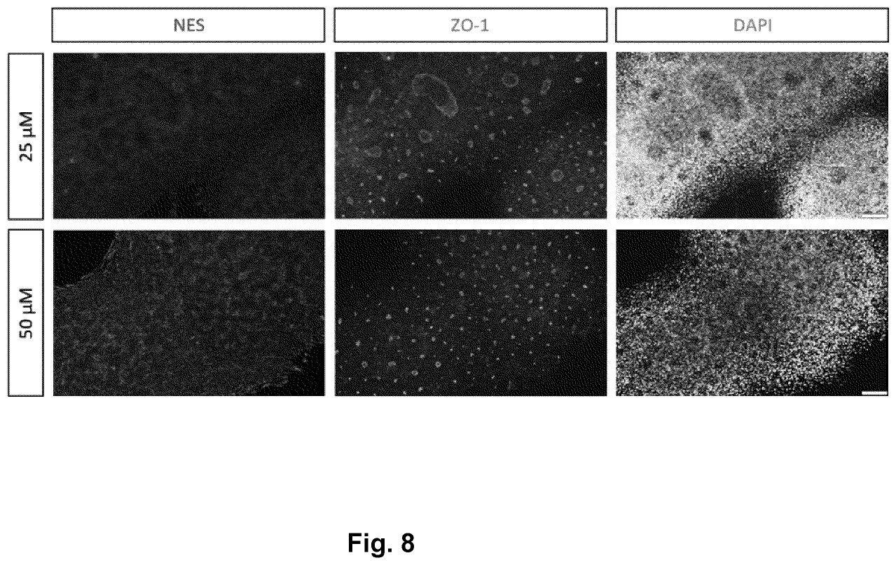

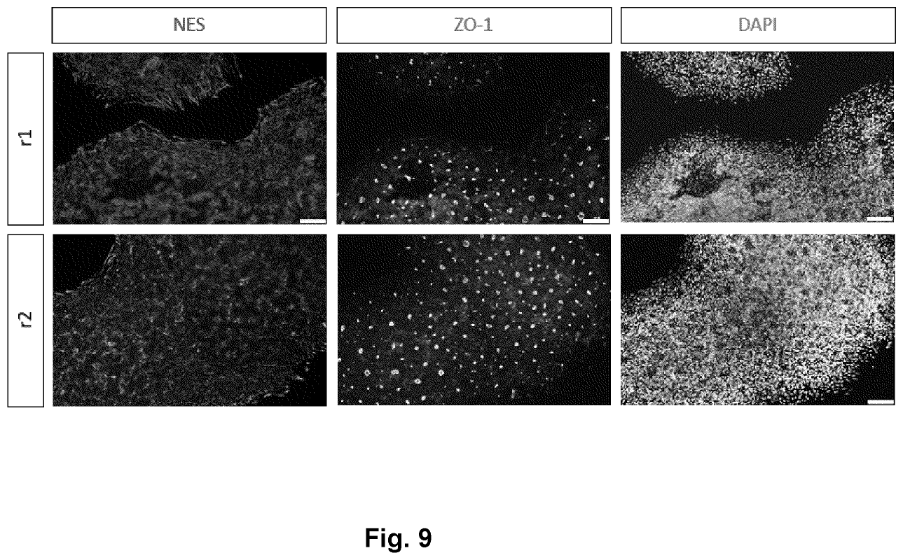

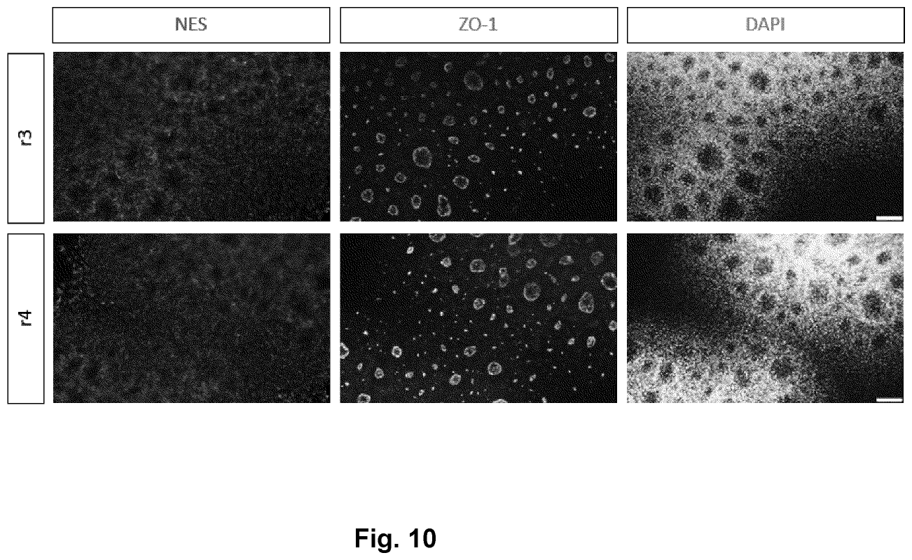

With the specific cell hESC line used here, the effect of RepSox seems to be superior for the formation of neural rosette structures compared to GW (FIG. 6). As shown in FIGS. 7 and 8, concentration of 25 and 50 μM of RepSox were the optimal to obtain an homogeneous population of neural rosettes with NES positive cells and ZO1 located in the center of each rosette. Lower concentrations had a less clear effect. FIGS. 9 and 10 showed four independent experiments (r1-4) to generate neural rosettes with RepSox at 50 μM, indicating the reproducibility of the described method.

In conclusion, NSCs can be produced efficiently with the single SMAD inhibitor RepSox in a highly effective and reproducible way. The SMAD inhibitor GW788388 might also be used.

### Example 2: Estimation of NECS Size to Produce Neural Rosettes

For the four independent experiments shown in FIGS. 9 and 10, NECS were collected at day 6, gently resuspended and a sample of 200 μL NECS suspension culture was diluted in 200 μL PBS. Diameter, size distribution, NECS count and circularity was determined. For example, we used Biorep® Islet Counter and its associated software (Biorep Technologies). BioRep® quantifies islets by a high-resolution image, which is subjected to Digital Image Analysis by which the individual islet's area is calculated. The calculated area is then used for estimating several characteristics of the islets including the individual diameters (d=2(A/π)^{1/2}) and the size distribution of the sample by a 50 μm increment group classification (Buchwald et al., 2016).

Prior to measuring, the sample was moved in figures of eight to distribute the aggregates allowing optimal detection. Additionally, if all NECS were not automatically detected by the software, clearly left out NECS were added to the data with the selection tool.

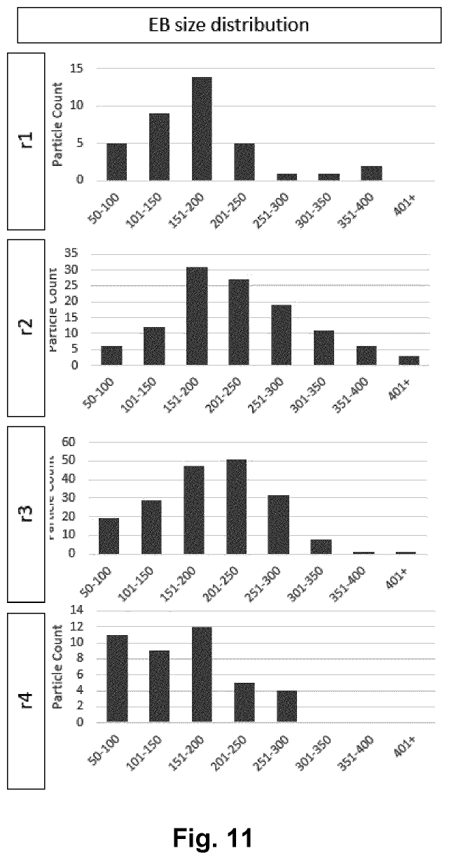

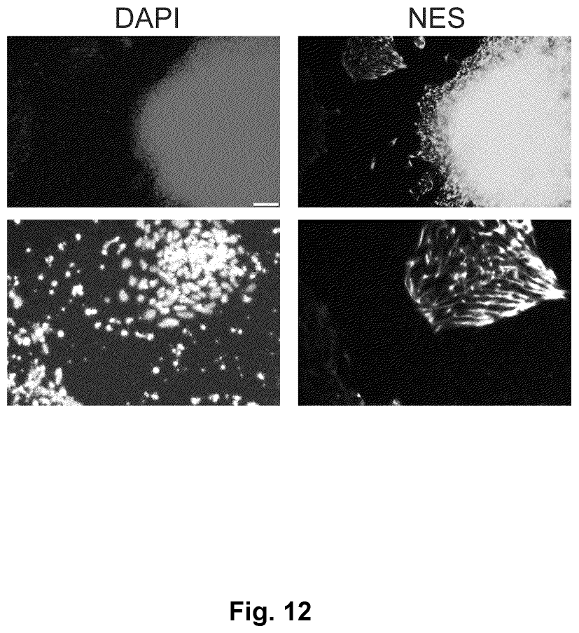

Homogeneity of NECS was examined by sorting the NECS diameters into 50 μm bins and evaluating the figurative distribution (FIG. 11). As shown in the graphs, NECS diameter smaller than 400 μm had a similar impact in the formation of neural rosettes where all cells were positive for NES. Bigger diameter, for example obtained by static culture, had a negative effect and produce fraction of NES negative cells (FIG. 12).

In conclusion, the diameter size of the NECS has a strong and direct effect of the cell purity, being smaller than 400 μm an ideal size to obtain a high homogeneous population of NSCs.

### Example 3: NSC Line Expansion and Establishment

At day 10, neural rosettes were perfectly defined. Cells were washed with PBS^{-/-}, treated with preheated TrypLE™ Select (37° C.) and incubated at 37° C. for 5-10 min. Cells were gently resuspended in preheated defined trypsin inhibitor (DTI from Gibco) and transferred to “wash media” consisting of DMEM/F12 GlutaMAX™ supplement (Gibco™) supplemented with 20 U/mL Penicillin-streptomycin (Gibco), 1% N-2 Supplement (Gibco™) and 10 μM ROCKi (Y-27632), which was centrifuged 1200 rpm for 3 min. After centrifugation the supernatant was removed, and the pellet was resuspended in neural expansion medium consisting of DMEM/F12 GlutaMAX™ supplement (Gibco™) supplemented with 20 U/mL Penicillin-streptomycin (Gibco), 1% N-2 Supplement (Gibco™), 10 μg/L bFGF, 10 μg/L EGF, 1‰ B-27 Supplement (Gibco™), supplemented with 10 μM ROCKi (Y-27632). The cell suspension was then seeded in culture plates coated with 1:50 natural mouse laminin (L2020-1MG, Sigma-Aldrich, 1 mg/ml).

This process was repeated once per day, for the following two to four days. Hereafter a milder dissociating method was performed, where cells were just washed with TrypLE™ Select and incubated for 2-4 min. at room temperature and then resuspended in DTI. The remaining steps followed the same method as described above. For every split NSCs were distributed in wells, which approximately corresponded to the double culture area, i.e. a 1:2 split. NSCs were not split before forming rosettes. Usually, it takes around two to three days for the NSCs to form rosettes. On days, where NSCs were not split, media was removed, and freshly prepared media was added without ROCKi (Y-27632).

Passage numbering was initiated when culturing was expanded to a 6-well format, i.e. after 5 splits over 9 days in this case.

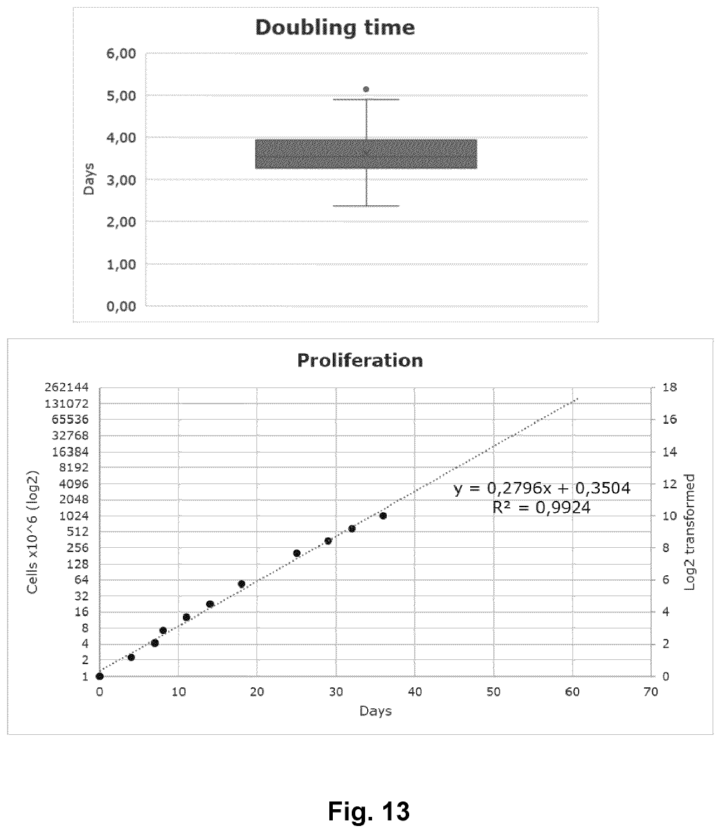

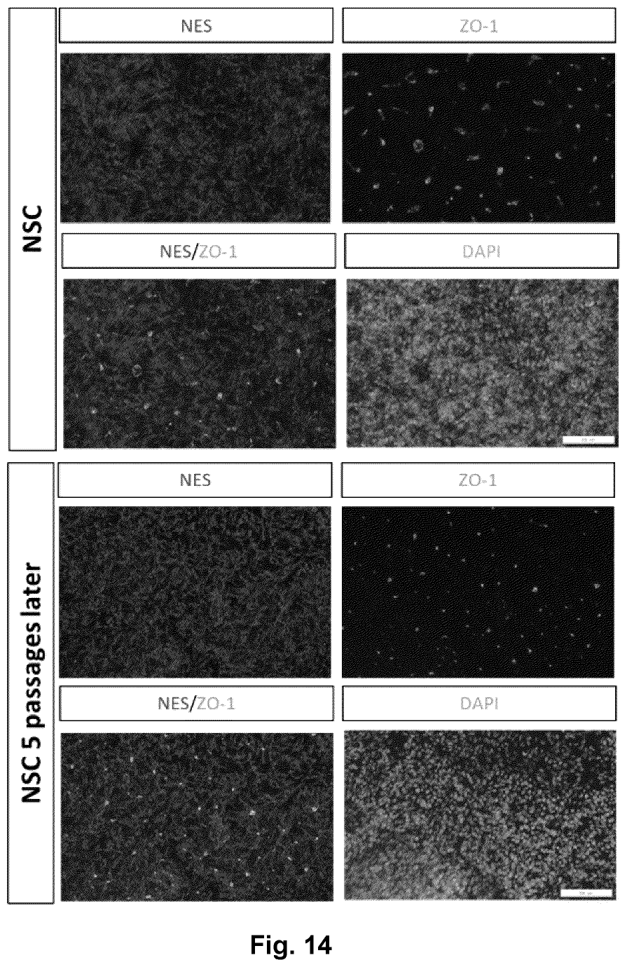

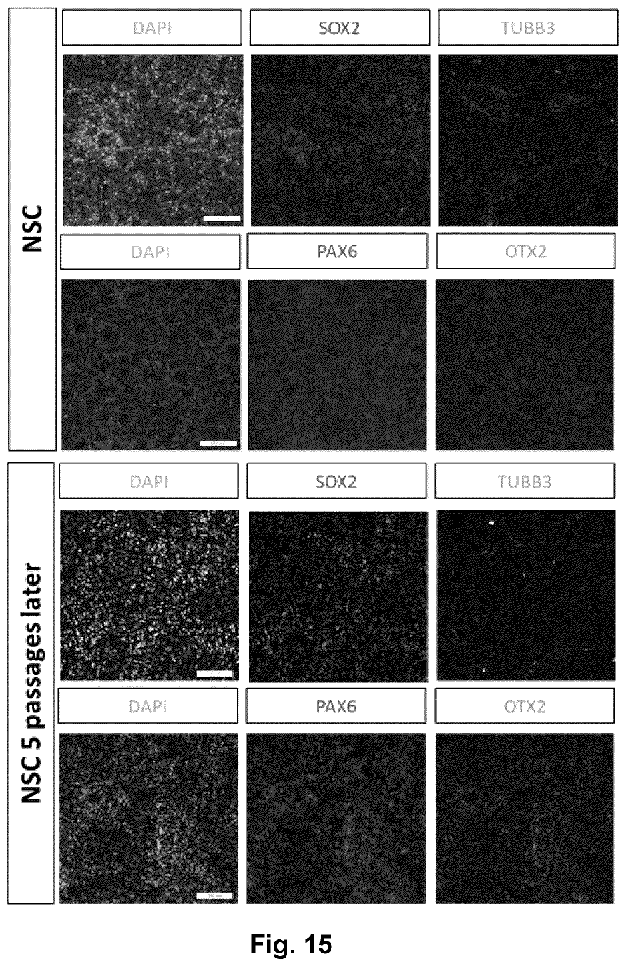

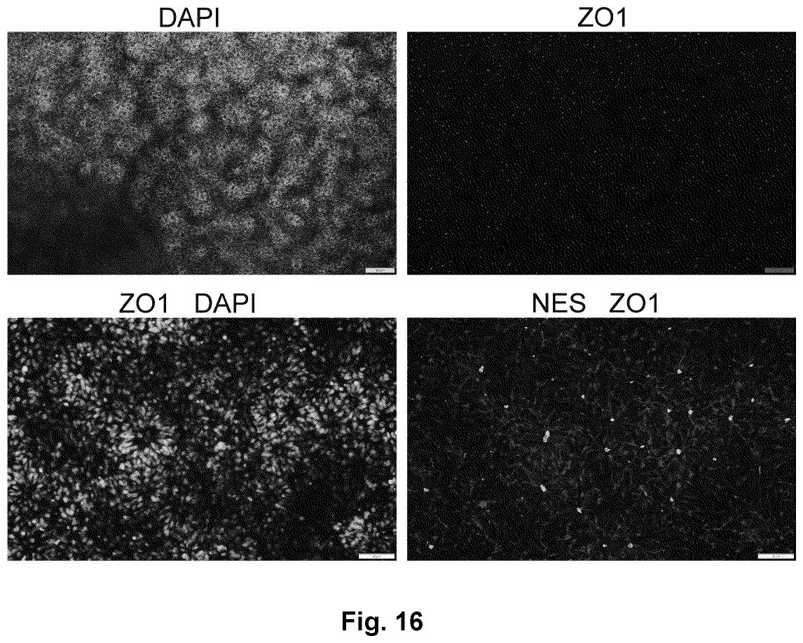

Following the successful rosette formation at day 10, cells were dissociated and replated for the NSC line establishment. NSCs were expanded over 36 days, and proportionally expanding from 1×10⁶ cells to more than 1×10⁹ cells with a doubling time of around 3-4 days, indicating continuous expandability (FIG. 13). After dissociation, the NSCs retained their rosette-forming tendency and sustained the expression of NSC markers NES, PAX6, OTX2 and SOX2 (FIG. 14-15). All cells were SOX2 positive. The typical rosette-like structures with pronounced central/lumen localization of ZO-1 were retained after several passages, where multiple tiny and uniform rosettes were seen (FIGS. 14 and 15, comparison with passage number 5). The capacity of these NSC to retain their rosette-forming tendency after dissociation can be seen also in FIG. 16, corresponding to passage number 12. The NSCs form multiple neural rosettes positive for NES and with ZO1 location in the lumen (FIG. 16). In addition, NSCs also continuously expressed neuronal markers, including the NSC markers PAX6 and SOX2.

For cryopreservation, cells are dissociated and isolated as described above. Cells are then resuspended in STEM-CELLBANKER® (Zenoaq) and stored at −80° C. for 24 hours before they are transferred vials to a N2 tank.

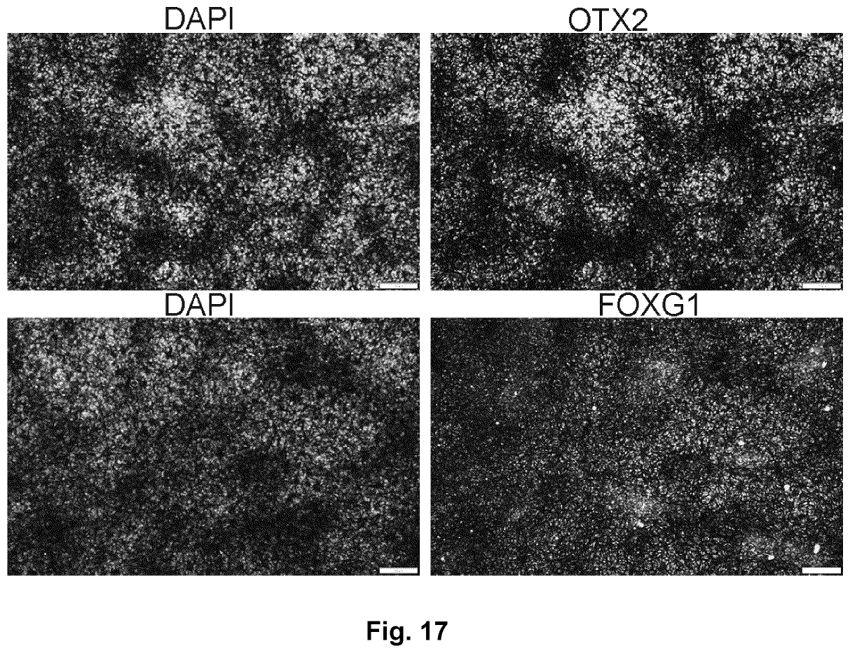

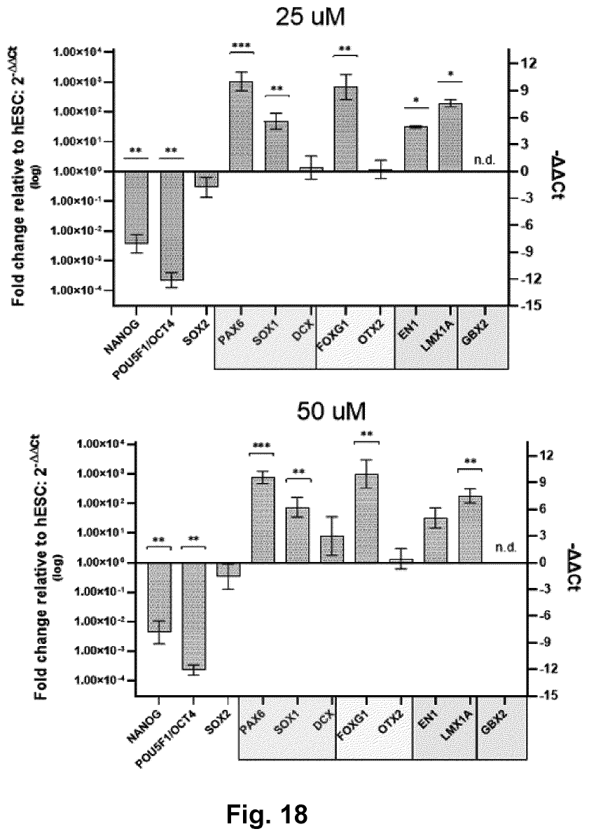

The NSC were still positive at passage 12 for the anterior markers FOXG1 and OTX2 (FIG. 17), indicating anterior (forebrain) identity. This identity was also analyzed at mRNA level, comparing two lines generated with RepSox at 25 and 50 μM (FIG. 18).

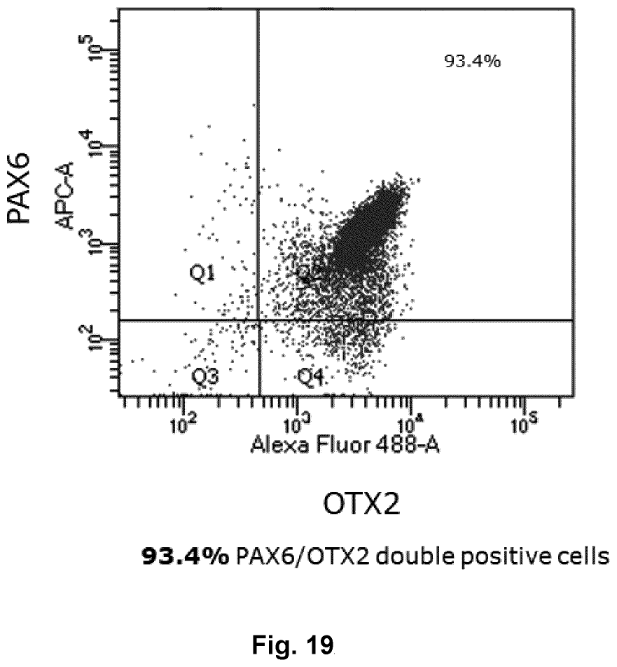

Flow cytometry analyses showed that more than 80% of the cells generated with this method were double positive for PAX6 and OTX2 markers at passage 3 (FIG. 19).

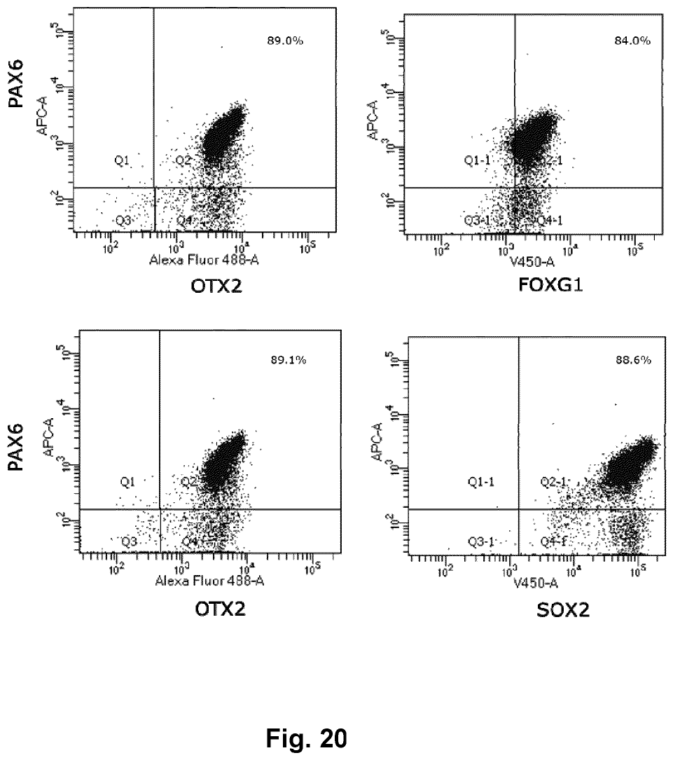

Flow cytometry analyses showed that more than 80% of the cells generated with this method were double positive for PAX6/OTX2, PAX6/FOXG1, PAX6/SOX2 markers at passage 8 (FIG. 20).

The collected data indicates that a pure and expandable cell line, which retain key NSC characteristics, can be generated following our protocol, retaining high percentage, more than 80%, for double positive PAX6/OTX2 and PAX6/SOX2 or triple positive PAX6/OTX2/SOX2

### Example 4: Comparison of Different SMAD Inhibitors

Table 1 shows the score of the different compounds and concentrations tested. The parameters measured were cell death, rosette formation, a monolayer formation in 2D, thickness of the epithelium formed in the rosette structure, and the absence of side population (all cells being NES and OTX2 positive), i.e. purity of the cell population

The following parameters were assessed and explained in further details in the following:

Cell death: Monitored by DAPI (4′,6-diamidino-2-phenylindole) staining. Pyknosis, or karyopyknosis, is the irreversible condensation of chromatin in the nucleus of a cell undergoing necrosis or apoptosis. After visual inspection under the fluorescent microscope, a score of 0 represent high number of pyknotic nuclei and a score of 3 a low number of pyknotic nuclei.

Rosette numbers: Monitored by DAPI, ZO1 and NES stainings. After visual inspection under the fluorescent microscope, a score of 0 represent low number of neural rosettes nuclei and a score of 3 a high number of rosettes.

Monolayer formation: Monitored by brightfield and DAPI staining. After visual inspection under the microscope, a score of 3 represent a flat monolayer of cells after NECS attachment.

Thickness of the rosette columnar epithelium: Monitored by DAPI staining. After visual inspection under the fluorescent microscope, a score of 3 represent a broader, thicker columnar epithelium of the neural rosette.

Side population: Monitored by specific stainings of NES and OTX2. After visual inspection under the fluorescent microscope, a score of 6 represents no detection of NES negative cells. Only in conditions without NES negative cells, the OTX2 population was evaluated. In those cases, a score of 3 represents no detection of OTX2 negative cells.

The following compounds and concentrations were tested and assessed according to the aforementioned parameter:
SB431542 10 μM
SB431542+LDN 10 μM+10 μM
GW 788388, concentration 10 ng/ml and 0.1 ng/ml
RepSox, doses 25 μM and 0.25 μM
SB525334, doses 10 μM and 0.1 μM
LY2157299, dose 10 μM
TEW-7197, dose 10 μM
LY2109761, dose 2 μM
Control: Non-treated with any SMAD inhibitor

### Example 5: Exosome Collection

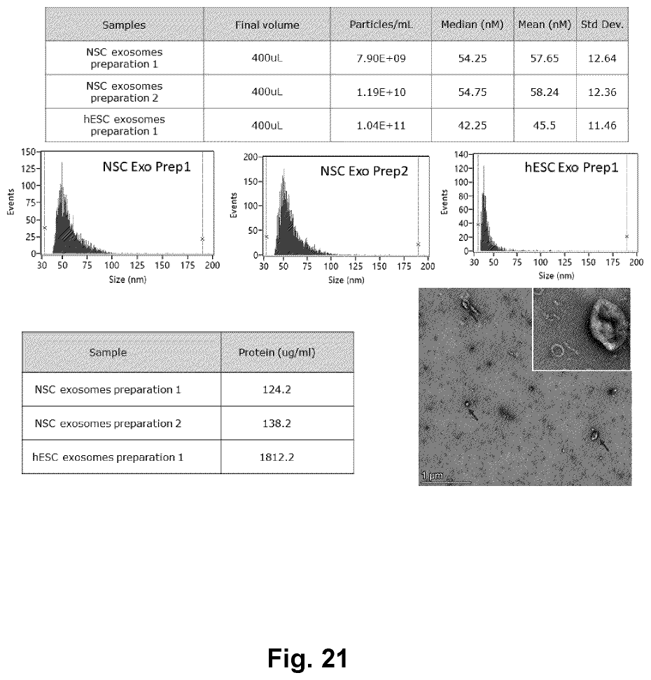

Supernatants from a population of at least 80% of triple positive NSCs for PAX6/OTX2/SOX2 and hESCs were collected and centrifuged for 10 min at 1500 g at 4 C to remove cells. The pellet was discarded, and the supernatant transferred to a new collection tube and stored at −80 C until ultracentrifugation for purification of exosomes. FIG. 21 shows the analyses of the exosomes present in supernatant collected from both hESC and NSC. Size, protein content and number of particles differs between exosomes produced by hESC and by NSC. The structure of exosomes produced by NSC is imaged by electron microscopy. This data indicated that a population of at least 80% of triple positive NSCs for PAX6/OTX2/SOX2 can generate exosomes.

TABLE 1
Comparison

| | | | | | No Side | No Side | |
|---|---|---|---|---|---|---|---|
| | Cell | | Monolayer | Thickness | Population | Population | Final |
| Compound | death | Rosette | formation | Epithelium | (NES Neg) | (OTX2 Neg) | Score |
| RepSox | 3 | 3 | 3 | 2 | 6 | 3 | 20 |
| LY2157299 | 3 | 3 | 2 | 3 | 6 | 0 | 17 |
| TEW-7197 | 0 | 2 | 2 | 2 | 6 | 3 | 15 |
| RepSox Low | 3 | 3 | 3 | 3 | 0 | N/A | 12 |
| GW788388 | 3 | 3 | 3 | 3 | 0 | N/A | 12 |
| SB525334 | 0 | 3 | 2 | 2 | 4 | N/A | 11 |
| SB431542 | 3 | 3 | 2 | 2 | 0 | N/A | 10 |
| SB525334 Low | 2 | 3 | 2 | 3 | 0 | N/A | 10 |
| GW788388 Low | 2 | 2 | 3 | 2 | 0 | N/A | 9 |
| SB431542/LDN | 0 | 0 | 0 | 0 | 6 | 3 | 9 |
| LY2109761 | 0 | 0 | 0 | 0 | 6 | 3 | 9 |
| Control | 0 | 0 | 0 | 0 | 0 | N/A | 0 |

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method for obtaining neuroectodermal cells from pluripotent stem cells (PSCs) comprising the steps of:
contacting said PSCs with ROCKi and a single SMAD inhibitor in suspension culture,
allowing said PSCs in suspension to spontaneously form tridimensional cell aggregates,
allowing said tridimensional cell aggregates to differentiate into neuroectodermal spheres with a diameter of less than about 500 μm in dynamic cell culture suspension,
allowing said neuroectodermal spheres to form neural rosettes, wherein said neural rosettes comprise neuroectodermal cells.

2. The method according to claim 1, wherein said neural rosettes are allowed to be maintained and expanded into neural stem cell (NSC) lines.

3. The method according to claim 1, where said neuroectodermal cells are neural stem cells.

4. A method for obtaining an in vitro neural stem cells, comprising the steps of:
dissociating the PSCs into single cells,
contacting said PSCs with ROCKi and a single SMAD inhibitor in suspension culture,
allowing the PSCs in suspension to spontaneously form tridimensional cell aggregates
allowing said tridimensional cell aggregates to differentiate into neuroectodermal spheres with a diameter of less than 500 μm in dynamic cell culture suspension,
plating the NECS-containing neuroectodermal cells on a substrate or optionally dissociating the NECS-containing NSCs,
allowing the NSCs to form neural rosettes and maintaining and expanding the NSCs to establish the NSC lines, without the need of manual picking and isolation.

5. The method according to claim 1, wherein the single SMAD inhibitor is RepSox or GW788388.

6. The methods according to claim 5, wherein RepSox is in the concentration of from about 20 μM to about 60 μM or wherein GW788388 is in the concentration from about 0.1 ng/ml to about 150 ng/ml.

7. The method according to claim 4, where said neural stem cells are at least 80% double positive for OTX2/PAX6 or SOX2/PAX6.

8. The method according to claim 4, where said neural stem cells are at least 80% triple positive for OTX2/PAX6/SOX2.

9. The method according to claim 4, where said neural stem cells are at least 80% quadruple positive for OTX2/PAX6/SOX2/FOXG1.

10. A method for producing extracellular vesicles comprising using the neural stem cell (NSC) lines obtained according to claim 4.

11. The method of claim 10, wherein said extracellular vesicles are exosomes.

12. A method of treating a neurodegenerative disorder comprising administering the exosomes of claim 11.

13. A highly pure population of neuroectoderm cells obtained by the method according to claim 1.

14. A method of obtaining neural or glia cells, comprising differentiating the highly pure population of neuroectoderm cells of claim 13.

15. A method of treating a neurodegenerative disorder, comprising transplanting said neural or glia cells of claim 14 into a subject in need thereof.

16. The method according to claim 4, wherein the single SMAD inhibitor is RepSox or GW788388.

17. The methods according to claim 16, wherein RepSox is in the concentration of from about 20 μM to about 60 μM or wherein GW788388 is in the concentration from about 0.1 ng/ml to about 150 ng/ml.

18. The method according to claim 3, where said neural stem cells are at least 80% double positive for OTX2/PAX6 or SOX2/PAX6.

19. The method according to claim 3, where said neural stem cells are at least 80% triple positive for OTX2/PAX6/SOX2.

20. The method according to claim 3, where said neural stem cells are at least 80% quadruple positive for OTX2/PAX6/SOX2/FOXG1.

21. The method according to claim 12, wherein the neurodegenerative disorder is stroke, traumatic brain injury or Alzheimer's disease.

22. The method according to claim 15, wherein the neurodegenerative disorder is stroke, traumatic brain injury or Alzheimer's disease.
